SEQUENCE LISTING (28 sequences)

SEQ ID NO: 1 - protein, 289 aa, Arthrobacter globiformis
  source 1..289: note = AglEpi; NCBI Accession No. BAW27657.1
MKIGCHGLVWTGHFDAEGIRYSVQKTREAGFDLVEFPLMDPFSFDVQTAKSALAEHGLAASASLGLSDATDVSSEDPAVVKAGEELLNRAVDVLAELGATDFCGVIYSAMKKYMEPATAAGLANSKAAVGRVADRASDLGINVSLEVVNRYETNVLNTGRQALAYLEELNRPNLGIHLDTYHMNIEESDMFSPILDTAEALRYVHIGESHRGYLGTGSVDFDTFFKALGRIGYDGPVVFESFSSSVVAPDLSRMLGIWRNLWADNEELGAHANAFIRDKLTAIKTIELH

SEQ ID NO: 2 - protein, 292 aa, Brachyspira suanatina
  source 1..292: note = BsuEpi1; NCBI Accession No. WP_048595324.1
MKYGIYYSYWEESWGADFVKYIEKVSKLGFDVLEINASALFDYSDNKINEVKKIINDNNIILTAGYGPIAEHNIGNINTRNKALDWYKKVFSVMQKLDIHIICGAIYSYWPVDYSKKINKEEDLKMSIEGMKLLANIAKEYNVNICCEVLNRFENYLLNTAKEAVAFVKEVGFDNVKVMLDTFHMNIEEESFANAIKEAGKYLGHFHLGEPNRRVPGEGRMPWNEILDALISIDYKGYAVMEPFVKRGGEVETDIKIWRDLVADTSEEKLDNAAKNSVHFLKTLEKIRRELL

SEQ ID NO: 3 - protein, 289 aa, Clostridiaceae bacterium
  source 1..289: note = CbaEpi17; NCBI Accession No. NLW02352.1
MKYGIYYAYWEKEWRGDFIPYIEKVKKLGFDVLEVACGDFHNQPDSYFHALRDAAKEYDITLTGGYGPRAEHNLASKDPAVVENALAFYSKIFSKMDMAGIRSIGGALYSYWPVDYSRDPDKEGDLERSIRGMQRLADIAARYGITLNMEVLNRFEGYLINDAREGLSYIRAVDKSNVKLMLDTFHMNIEEDSLTEPILLAGKDLGHLHVGEPNRKPPREGRMPWKEIGQALRQIGYEGAVVMEPFVTMGGQVGKDIRVWRDISGGATRSDLDRDAAKSVSFLRSMFEG

SEQ ID NO: 4 - protein, 289 aa, Clostridiaceae bacterium
  source 1..289: note = CbaEpi18; NCBI Accession No. NLI38423.1
MKYGIYYAYWEKEWGGDFIPYVAKVKQLGFDVLEVACGDFHNQPDSYLHALRETAKENDIILTGGYGPRAEHNLASPDPAVVENALAFYSEIFRKMDIADIRSIGGALYSYWPIDYNKELDKDGDLERSIQGMRKLADIAGRYGITLNMEVLNRFEGYLINDVREGLAYVRAVDKPNVKLMLDTFHMNIEEDSLIEPILLAGKDLGHVHVGEPNRKPPREGRMPWKEIGVALREIGYDGAVVMEPFVTMGGQVGKDIRIWRDISQGATENDLDRDAAKSVAFLRSAFEE

SEQ ID NO: 5 - protein, 291 aa, Nakamurella deserti
  source 1..291: note = NdeEpi1; NCBI Accession No. WP_111768618.1
MPSLIGCHALVWTGEFDAAGITSSVERTKAAGFDLVEFPLMDPYTFDAATARKALGDADLTGSASLGLPEHADISSEDPAVVAAGVDLLRTALDRVAEMGGTRLCGVLYGSMRKHMAPLSERGRANSVAAIRDLAGRAAAVGVHLSLEVVNRYETNVFNTGRGALAYLDAVADTNVSVHLDTYHMNIEESDLFQPFLDTSERLGYVHIGESHRGYLGTGSVDFESTFRALARTGYDGPIVFESFSSAVVSPTLSNTLGIWRNLWTDSEDLAAHANRFIRDHIAAVRTIDLH

SEQ ID NO: 6 - protein, 289 aa, Labedella phragmitis
  source 1..289: note = LphEpi1; NCBI Accession No. WP_128493859.1
MQIGVHALVFTGTFDRDGISLAVEKTAAAGFDLIEFPLMDPFAFDREHAAKELAAHGIAATASLGLSDATDLTSDDPAVRAAGERLLEECLDIVHALGATKLCGVLYSAMKKYVTPPTAEGRAASAAAIGRLAEKAADLGIDLSLEVVNRYETNLINTGRQALGFLDEIGRGNVFVHLDTYHMNIEESDLIQPVLDVDRRLGYVHIGESHRGYLGSGTVDFDSFFRGLARVGYDGPVVFESFSSTVVSATLSNTLGVWRNLWDDGDDLAAHANRFIRDGLRTVETIALH

SEQ ID NO: 7 - protein, 289 aa, Mycetocola zhujimingii
  source 1..289: note = MspEpi3; NCBI Accession No. WP_108392186.1
MLIGAHALVFSGSFDAAGLTLAIEKTKQAGFDLIEIPLMDPFHFDRMLAARLLQENELAVSASLGLSEATDLTSNDPDVVARGERMLEECLEIVHAMGGSDLCGVIYSAMKKYMAPATPAGRASSAAALARLASRAEGLGIRLSLEVVNRYETNLMNTGRQALAFLDEVGHDNVSVHLDTYHMNIEESDLYTPVLDVGNRLGYAHVGESHRGYLGSGTVDFGSFFRGLARIGYDGPVVFESFSSAVVNEELSRMLGIWRNLWDDPDDLGAHANRFIRTQLRAVETIALH

SEQ ID NO: 8 - protein, 291 aa, Oribacterium sinus
  source 1..291: note = OsiEpi1; NCBI Accession No. WP_183684385.1
MKYGIYYAYWEKEWTGDYFKYIDKVKELGFDILEISCGAFKKDYTTDEQLLRIGEYAKEKGIILTAGYGPQKDENLASPDEAVQKNAIEFFKETLRKLHLMNIHILGGGLYSYWPVDFNSDINKERDFETSVKNMRLIAKYAEEYDITLGMEVLNRFEGYLINTCDECLRYIDAVGNNRVGVMLDTFHMNIEEDNIGDAIRKAGDKLVHFHMGERNRKVPGKGTMPWAEIGQALRDIHYQHAAVMEPFVLQGGKVGQDIKIWRNLLPDTSEAKLDADAKHSCEFVRHVFEL

SEQ ID NO: 9 - protein, 289 aa, Mycetocola sp. CAN_C7
  source 1..289: note = MspEpi4; NCBI Accession No. WP_196832999.1
MLIGAHALVFTGSFEQAGLTTAIEKTKQAGFDLIEIPLMDPANFDRMLAARLLRDNDLAVSASLGLSEATDLTSGDPAVVAAGERMLEECIDIVQAMGGTRLCGVIYSAMKKYMEPVTASGRASSAAALGRLASRAEDAGVHLSLEVVNRYETNVMNTGRQALGFLDEIGHSNVSVHLDTYHMNIEESDLYTPVLDVDTRLGYVHIGESHRGYLGSGTVDFGSFFRGLARVSYDGPVVFESFSSAVVNQDLSRMLGIWRNLWDDSDDLAAHANRFIRDQLRAVESISLH

SEQ ID NO: 10 - DNA, 867 bp, synthetic construct
  misc_feature 1..867: note = Artificial Sequence
  misc_feature 1..867: note = AglEpi
  source 1..867: mol_type = other DNA
atgaaaattggctgccatggcttagtgtggacaggacattttgatgcggaaggcatccgttattcagtccaaaaaacacgcgaagcaggctttgatcttgtggaatttcctcttatggatccgtttagctttgatgttcaaacagcgaaatcagcacttgcagaacatggccttgcagcgtctgcgagcctgggactgtcagatgcaacggatgtctcttctgaagatccggcggtggtcaaagcgggagaagaattacttaatcgtgcagtcgatgtgcttgcggaattaggagcaacggatttttgcggagttatctatagcgcaatgaaaaaatatatggaacctgcgacagcagcaggccttgcaaattctaaagcggcggtcggccgggtcgcggatcgcgcgtcagatttaggcatcaatgtctcactggaagtggtcaatcggtatgaaacgaatgtccttaatacaggacgccaagcgctggcgtatcttgaagaacttaatagacctaatctgggcattcatttagatacgtatcatatgaatatcgaagaaagcgatatgtttagccctatcctggatacggcagaagcacttcggtatgtccatattggagaatcacatagaggctatttaggaacgggctcagtcgattttgatacgttttttaaagcgttgggacgcattggctatgatggcccggttgtgtttgagagcttttctagctcagttgtcgcacctgatctttctcgcatgctgggcatctggcgtaatctgtgggcagataatgaagaactgggagcacatgcaaatgcgtttattcgtgataaactgacggcgattaaaacaatcgaacttcat

SEQ ID NO: 11 - DNA, 876 bp, synthetic construct
  misc_feature 1..876: note = Artificial Sequence
  misc_feature 1..876: note = BsuEpi1
  source 1..876: mol_type = other DNA
atgaaatatggcatctattatagctattgggaagaatcttggggcgcagattttgttaaatatatcgaaaaagtttctaaacttggctttgatgttctggaaatcaatgcaagcgcactgtttgattatagcgataataaaatcaatgaagttaaaaaaatcatcaatgataataatatcatcctgacggcaggctatggcccgatcgcagaacataatatcggcaatatcaatacacgcaataaagcattagattggtataaaaaagtttttagcgttatgcaaaaacttgacatacatatcatctgcggagcaatttatagctattggccggttgattattctaaaaaaattaataaagaagaagatcttaaaatgtctattgaaggcatgaaactgctggcaaatatcgcaaaagaatataatgttaatatctgctgcgaagttctgaatcgctttgaaaattatttactgaatacagcaaaagaagcagttgcatttgttaaagaagttggctttgataatgttaaagttatgctggatacgtttcatatgaatatcgaagaagagtcgtttgcaaatgcaatcaaagaagcaggcaaatatttaggacattttcatttaggcgaacctaatagacgcgttccgggcgaaggacgcatgccgtggaatgaaatcctggatgcactgatcagcatcgattataaaggctatgcagttatggaaccgtttgttaaacgcggaggagaagttgaaacggacataaaaatctggagagatctggttgcagatacgagcgaagaaaaacttgataatgcagcaaaaaattcagttcattttcttaaaacactggaaaaaattcgccgcgaactgctg

SEQ ID NO: 12 - DNA, 867 bp, synthetic construct
  misc_feature 1..867: note = Artificial Sequence
  misc_feature 1..867: note = CbaEpi17
  source 1..867: mol_type = other DNA
atgaaatatggcatctattatgcatattgggaaaaagaatggagaggcgattttatcccgtatatcgaaaaagttaaaaaactgggctttgatgttctggaagttgcatgcggagattttcataatcaaccggattcttattttcatgcactgcgcgatgcagcaaaagaatatgacataacactgacgggaggctatggccctcgcgcagaacataatttagcatctaaagatccggcagttgttgaaaatgcacttgcattttattctaaaatcttttctaaaatggatatggcaggcatccgctcaatcggcggcgcactgtatagctattggccggttgattatagccgcgatccggataaagaaggagatttagaacgctcaattagaggaatgcaacgccttgcagacatcgcagcacgctatggaattacacttaatatggaagttcttaatcgctttgaaggctatctgatcaatgatgcacgcgaaggactgtcttatatcagagcagttgataaatctaatgttaaactgatgctggatacgtttcatatgaatatcgaagaagatagcctgacggaaccgatcctgctggcaggcaaagatctgggacatttacatgttggcgaacctaatcgcaaacctcctagagaaggccgcatgccgtggaaagaaattggacaagcacttagacaaatcggctatgaaggagcagttgttatggaaccgtttgttacgatgggcggacaagttggcaaagatattcgcgtttggcgcgacataagcggcggagcaacacgctcagatcttgatagagatgcagcaaaatcagttagctttctgcgctcaatgtttgaagga

SEQ ID NO: 13 - DNA, 867 bp, synthetic construct
  misc_feature 1..867: note = Artificial Sequence
  misc_feature 1..867: note = CbaEpi18
  source 1..867: mol_type = other DNA
atgaaatatggcatctattatgcatattgggaaaaagaatggggcggagattttattccgtatgttgcaaaagttaaacaattaggctttgatgttcttgaagttgcatgcggagattttcataatcaaccggattcttatttacatgcactgcgcgaaacagcaaaagaaaatgacatcatcctgacgggcggctatggccctagagcagaacataatttagcatcaccggatccggcagttgttgaaaatgcacttgcattttatagcgaaatctttcgcaaaatggatattgcagacatacgctcaattggaggcgcactgtatagctattggccgatcgattataataaagaactggataaagatggcgatcttgaacgctcaattcaaggaatgcgcaaacttgcagatatagcaggacgctatggaattacacttaatatggaagttcttaatcgctttgaaggctatctgatcaatgatgttcgcgaaggactggcatatgttagagcagttgataaacctaatgttaaactgatgctggatacgtttcatatgaatatcgaagaagattctcttatcgaaccgatcctgctggcaggcaaagatctgggacatgttcatgttggcgaacctaatcgcaaacctcctcgcgaaggccgcatgccgtggaaagaaatcggcgttgcactgagagaaatcggctatgatggagcagttgttatggaaccgtttgttacgatgggaggacaagttggcaaagacataagaatctggcgcgatatttcacaaggagcaacggaaaatgatttagatagagatgcagcaaaaagcgttgcatttctgcgctcagcatttgaagaa

SEQ ID NO: 14 - DNA, 873 bp, synthetic construct
  misc_feature 1..873: note = Artificial Sequence
  misc_feature 1..873: note = NdeEpi1
  source 1..873: mol_type = other DNA
atgccgtcacttatcggatgccatgcattagtttggacaggcgaatttgatgcagcaggcatcacgagctctgttgaacgcacaaaagcagcaggctttgatctggttgaatttcctcttatggatccgtatacgtttgatgcagcaacggcaagaaaagcactgggagatgcagatcttacaggaagcgcaagcctgggacttccggaacatgcagatattagctcagaagatccggcagttgttgcagcaggagttgatttactgagaacggcattagatagagttgcagaaatgggaggaacacgcctgtgcggcgttctgtatggctcaatgagaaaacatatggcaccgctgtcagaacgcggacgcgcaaatagcgttgcagcaattcgcgatcttgcaggccgcgcagcagcagttggcgttcatctgagcctggaagttgttaatcgctatgaaacaaatgtttttaatacgggccgcggagcattagcatatctggatgcagttgcagatacgaatgttagcgttcatttagatacgtatcatatgaatatcgaagaaagcgatttatttcaaccttttcttgatacgagcgaaagacttggctatgttcatattggcgaatcacatagaggctatttaggaacgggctcagttgattttgaatcaacgtttcgcgcactggcacgcacgggctatgatggacctattgtttttgaatcgttttctagcgcagttgttagcccgacactgtctaatacacttggcatctggagaaatctgtggacggattctgaagatcttgcagcacatgcaaatcgctttatccgcgatcatatcgcagcagttcgcacaatcgatttacat

SEQ ID NO: 15 - DNA, 867 bp, synthetic construct
  misc_feature 1..867: note = Artificial Sequence
  misc_feature 1..867: note = LphEpi1
  source 1..867: mol_type = other DNA
atgcaaatcggagttcatgcactggtttttacgggaacgtttgatagagatggcatctctctggcagttgaaaaaacggcagcagcaggctttgatcttatcgaatttccgttaatggatccgtttgcatttgatagagaacatgcagcaaaagaacttgcagcacatggcatcgcagcaacggcatcactgggactgagcgatgcaacagatctgacgagcgatgatccggcagttcgcgcagcaggagaacgcttactggaagaatgcctggacatagttcatgcattaggcgcaacgaaactgtgcggagttctgtatagcgcaatgaaaaaatatgttacacctccgacggcagaaggccgcgcagcaagcgcagcagcaattggccgccttgcagaaaaagcagcagatttaggaatcgatttaagccttgaagttgttaatcgctatgaaacaaatcttattaatacgggccgccaagcattaggctttctggatgaaattggacgcggaaatgtttttgttcatttagatacgtatcatatgaatatcgaagaatcagatcttattcaaccggttctggatgttgatagacgcctgggctatgttcatatcggcgaatcacatagaggctatctgggctcaggaacagttgattttgatagcttttttcgcggacttgcacgcgttggctatgatggaccggttgtttttgaatcatttagctctacggttgttagcgcaacactgtctaatacacttggcgtttggagaaatctgtgggatgatggcgatgatcttgcagcacatgcaaatagatttattcgcgatggacttcgcacagttgaaacaattgcattacat

SEQ ID NO: 16 - DNA, 867 bp, synthetic construct
  misc_feature 1..867: note = Artificial Sequence
  misc_feature 1..867: note = MspEpi3
  source 1..867: mol_type = other DNA
atgcttatcggcgcacatgcattagtttttagcggcagctttgatgcagcaggacttacgttagcaatcgaaaaaacgaaacaagcaggctttgatcttatcgaaatccctcttatggatccgtttcattttgatagaatgctggcagcacgcttacttcaagaaaatgaacttgcagtttcagcatcactgggcctgagcgaagcaacagatctgacgtctaatgatccggatgttgttgcaagaggcgaacgcatgctggaagaatgcctggaaatcgttcatgcaatgggcggctcagatctgtgcggagttatctattctgcaatgaaaaaatatatggcaccggcaacacctgcaggccgcgcatctagcgcagcagcactggcacgccttgcatctcgcgcagaaggactgggtatccgcctgtctcttgaagttgttaatcgctatgaaacaaatttaatgaatacgggccgccaagcacttgcatttctggatgaagttggccatgataatgttagcgttcatttagatacgtatcatatgaatatcgaagaaagcgatctgtatacaccggttcttgatgttggcaatagattaggatatgcacatgttggagaatcacatagaggctatttaggctcaggaacggttgattttggttccttttttcgcggactggcacgcatcggctatgatggaccggttgtttttgaatcgttttctagcgcagttgttaatgaagaactgtcacgcatgctgggaatttggcgcaatctgtgggatgatccggatgatttaggagcacatgcaaatagatttatcagaacacaactgagagcagttgaaacgattgcattacat

SEQ ID NO: 17 - DNA, 873 bp, synthetic construct
  misc_feature 1..873: note = Artificial Sequence
  misc_feature 1..873: note = OsiEpi1
  source 1..873: mol_type = other DNA
atgaaatatggcatctattatgcatattgggaaaaagaatggacgggcgattattttaaatatatcgataaagttaaagaacttggctttgatattctggaaatctcttgcggcgcatttaaaaaagattatacgacggatgaacaactgctgcgcatcggcgaatatgcaaaagaaaaaggcatcatccttacagcaggctatggacctcaaaaagatgaaaatttagcatcaccggatgaagcagttcaaaaaaatgcaatcgaattttttaaagaaacacttcgcaaacttcatcttatgaatatccatatcctgggaggaggactgtatagctattggccggttgattttaattctgacataaataaagaacgcgattttgaaacgagcgttaaaaatatgagactgattgcaaaatatgcagaagaatatgatataacattaggcatggaagttcttaatcgctttgaaggctatctgatcaatacgtgcgatgaatgcttacgctatattgatgcagttggaaataatagagttggcgttatgttagatacgtttcatatgaatatcgaagaagataatatcggagatgcaattcgcaaagcaggagataaactggttcattttcatatgggcgaacgcaatcgcaaagttccgggcaaaggcacaatgccgtgggcagaaatcggccaagcactgagagatattcattatcaacatgcagcagttatggaaccgtttgttcttcaaggaggcaaagttggccaagatattaaaatctggcgcaatctgttaccggatacgtcagaagcaaaactggatgcagatgcaaaacatagctgcgaatttgttagacatgtttttgaactt

SEQ ID NO: 18 - DNA, 867 bp, synthetic construct
  misc_feature 1..867: note = Artificial Sequence
  misc_feature 1..867: note = MspEpi4
  source 1..867: mol_type = other DNA
atgcttattggagcacatgcactggtttttacgggctcttttgaacaagcaggactgacgacggcaatcgaaaaaacgaaacaagcaggctttgatttaatcgaaattcctcttatggatccggcaaattttgatagaatgctggcagcacgcttactgagagataatgatcttgcagttagcgcatcactgggcctgtcagaagcaacggatttaacgtcaggcgatccggcagttgttgcagcaggagaacgcatgctggaagaatgcatcgatattgttcaagcaatgggaggaacacgcctgtgcggagttatctatagcgcaatgaaaaaatatatggaacctgttacggcatcaggacgcgcaagctctgcagcagcattaggccgccttgcatctagagcagaagatgcaggcgttcatctgagcttagaagttgttaatcgctatgaaacaaatgttatgaatacgggccgccaagcactgggctttctggatgaaattggccattctaatgttagcgttcatttagatacgtatcatatgaatatcgaagaaagcgatctgtatacaccggttctggatgttgatacgagacttggctatgttcatatcggcgaatcacatagaggctatttaggctcaggaacggttgattttggctcattttttcgcggacttgcacgcgtttcttatgatggaccggttgtttttgaaagtttttctagcgcagttgttaatcaagatctgtctcgcatgctgggaatttggcgcaatctgtgggatgattcagatgatcttgcagcacatgcaaatagatttattcgcgatcaacttcgcgcagttgaatcaatctcattacat

SEQ ID NO: 19 - protein, 289 aa, Oscillospiraceae bacterium
  source 1..289: note = OspEpi5; NCBI Accession No. MBR3355450.1
MKYGIYFAYWEKYWGADQVKYIKKVKELGFDILEVSGALLKNNTKERLAEIGQIAKDEGVILTCGYGPNASENLGSSDPDVVRNAISMYTDILQKMQVMDIHTFGGGIYSYWPVDYSKPIDKPGDWERSVKNVRTVGKIAEECGVDYCLEVLNRFEGYLINTCEECVKFVDEVDVPSVKVMLDTFHMNIEEDDMCDAIRLAGKRLGHFHTGENNRKVPGRGKFPWYDLGKALREIGYDKNVVMEPFVQMGGEVGSDIKVWRDISKGATPQQLDEDAAFSVAFLRKAFEG

SEQ ID NO: 20 - protein, 289 aa, Dorea sp.
  source 1..289: note = DspEpi3; NCBI Accession No. CDD07088.1
MKHGIYYAYWEKEWAADYLYYVEKVARLGFDLLEIGAAPLPEYSTDQIKALRDCASQNGIQLTAGYGPTYDHNMGSSDAGIRAGALEWYKRLFDVMEQLDIHLIGGALYGYWPVDFSNINKEEDWKRSVEGMHLLAPIAKEHDINLGMEVLNRFESHILNTAEEGVAFVKEVGQENVKVMLDTFHMNIEEESIGDAIRTAGNLLGHFHTGECNRMVPGKGRTPWREIGNALRDIEYDGTVVMEPFVSMGGQVGRDIHIWRDISRGASEAELDKDAKNAVAFQKYMLDWK

SEQ ID NO: 21 - protein, 308 aa, Oscillospiraceae bacterium
  REGION 1..295: note = OspEpi3; NCBI Accession No.
MKFGIYFAYWEREWNADQAKYIAKVKRLGFDGLELSCALLKNASDRELDELRRAAEAEGVTLTAGYGPAAEEDLASSDPAVTRHAVAVYTDLLKKLERLNIHTIGGGIYSYWPVDFSRPLDKAGDWARSVENVRKVGRIAGECGVNYCLECLNRFEGYLINTCAECRAFVEEVDVPAVKIMLDTFHMNIEEDDMTAAIRLAGDRLGHFHVGENNRRVPGKGGLPWYAVGEALRSIGYNGNVVMEPFVRPGGGVGRDIRIWRDLSAGADEAVLDADARGSVEFLRFVFSGPTGGRDPQRCAYGSQFNRI

SEQ ID NO: 22 - protein, 295 aa, Oscillospiraceae bacterium
  source 1..295: note = OspEpi10; NCBI Accession No. MBR2672832.1
MKYGIYFAYWEKYWDVDQVKYIKKVKDLGFDILEVSGALLKNNTRERLREIGEMAKAEGVILTCGYGPNAKEDLGSADPEVRKNAVAVYTDLLEKMRIMNIHTFGGGIYSYWPVDYSKPIDKEGDWARSVDGVRTVGKIAGECGVDYCLEVLNRFEGYLINTCEECMKFVDEVGEPAVKVMLDTFHMNIEEDDMIKAIRLAGDKLGHFHVGENNRKLPGKGRFPWREIGRALQDIGYQKNVVMEPFVQMGGEVGSDIKVWRDISGGATGEQLDRDAKDSVAFLRYAFEGTGGRLW

SEQ ID NO: 23 - protein, 288 aa, synthetic construct
MKYGIYYAYWEKEWGGDFIPYVAKVKQLGFDVLEVACGDFHNQPDSYLHALRETAKENDIILTGGYGPRAEHNLASPDPAVVENALAFYSEIFRKMDIADIRSIGGALYSYWPIDYNKELDKDGDLERSIQGMRKLADIAGRYGITLNMEVLNRFEGYLINDVREGLAYVRAVDKPNVKLMLDTFHMNIEEDSLIEPILLAGKDLGHVHVGEPNRKPPREGRMPWKEIGVALREIGYDGAVVMEPFVTMGGQVGKDIRIWRDISQGATENDLDRDAAKSVAFLRSAFE

SEQ ID NO: 24 - DNA, 870 bp, synthetic construct
  source 1..870: mol_type = other DNA
gtgaaatatggcatttattttgcatattgggaaaaatattggggcgcagatcaggtgaaatatattaaaaaagtcaaagaactgggctttgatattctggaagttagcggcgcgctgctgaaaaataatacaaaagaaagactggcggaaattggccaaattgcaaaagatgaaggcgtgattctgacatgcggctatggcccgaatgctagcgaaaatcttggcagcagcgatccggatgtggttagaaatgcgattagcatgtatacagatattcttcaaaaaatgcaagtgatggatattcatacgtttggcggcggcatttatagctattggccggtggattatagcaaaccgattgataaaccgggcgattgggaaagaagcgttaaaaatgtcagaacagttggaaaaattgcagaagaatgcggcgttgattattgcctggaagtgctgaatagatttgaaggctatctgattaatacatgcgaagaatgcgttaaatttgtggatgaagttgatgttccgagcgttaaagtgatgcttgatacatttcatatgaatattgaagaagatgatatgtgcgatgcgattagacttgcaggcaaaagactgggccattttcatacaggagaaaataatagaaaagtgccgggcagaggcaaatttccgtggtatgatcttggcaaagcacttagagaaattggctatgataaaaatgtggttatggaaccgtttgtgcaaatgggcggcgaagttggatcagatattaaagtttggagagatattagcaaaggagcgacaccgcaacaacttgatgaagatgcagcattttcagttgcgtttctgagaaaagcatttgaaggctaa

SEQ ID NO: 25 - DNA, 870 bp, synthetic construct
  source 1..870: mol_type = other DNA
gtgaaacatggcatttattatgcgtattgggaaaaagaatgggcagcagattatctgtattatgttgaaaaagttgcgagactgggctttgatctgctggaaattggcgcagcaccgttaccggaatattcaacagatcaaattaaagcactgagagattgcgcaagccaaaatggcattcagctgacagcaggctatggcccgacatatgatcataatatgggcagctcagatgcaggcattagagcaggcgcacttgaatggtataaaagactgtttgatgttatggaacaacttgatattcatttaattggcggagcgctgtatggctattggccggttgatttttcaaatattaataaagaagaagattggaaaagaagcgtggaaggcatgcatctgctggcgccgattgcaaaagaacatgatattaatcttggcatggaagttctgaatagatttgaatcacatattctgaatacagcagaagaaggcgtggcatttgttaaagaagttggccaagaaaatgttaaagttatgttagatacatttcatatgaatattgaagaagaaagcattggcgatgcgattagaacagcaggcaatcttctgggccattttcatacaggcgaatgcaatagaatggtgccgggcaaaggcagaacaccgtggagagaaattggcaatgcactgagagatattgaatatgatggcacagtcgttatggaaccgtttgtttcaatgggcggccaagttggcagagatattcatatttggagagatattagcagaggcgcatcagaagcggaactggataaagatgcaaaaaatgcagttgcatttcaaaaatatatgcttgattggaaataa

SEQ ID NO: 26 - DNA, 927 bp, synthetic construct
  source 1..927: mol_type = other DNA
gtgaaatttggcatttattttgcatattgggaaagagaatggaatgcagatcaagcaaaatatattgcaaaagttaaaagacttggatttgatggcctggaactgtcatgcgcgctgcttaaaaatgcaagcgatagagaactggatgaacttcgcagagcagcggaagcagaaggcgtcacactgacggcaggctatggcccggcggcggaagaagatctggcaagcagcgatccggcagttacacgccatgcggtggcagtgtatacagatcttctgaaaaaacttgaacgcctgaatattcatacaattggaggcggcatttatagctattggccggtggattttagcagaccgcttgataaagcgggagattgggctagaagcgttgaaaatgttagaaaagttggcagaattgcgggcgaatgcggagtgaattattgcctggaatgcctgaatcgctttgaaggctatctgattaatacatgcgcagaatgcagagcttttgttgaagaagttgatgttccggcagtgaaaatcatgctggatacatttcatatgaatattgaagaagatgatatgacggcggcaattagacttgcaggcgatagattaggccattttcatgtgggcgaaaataatcgcagagtgccgggcaaaggcggcctgccgtggtatgctgttggcgaagcgcttagaagcattggctataatggcaatgttgttatggaaccgtttgtgagaccgggcggcggcgtcggcagagatattagaatttggagagatctgagcgcaggcgcagatgaagcggtgctggatgcggatgcgagaggcagcgtggaatttctgagatttgtttttagcggcccgacaggcggcagagatccgcagagatgcgcgtatggcagccaatttaatagaatttaa

SEQ ID NO: 27 - DNA, 888 bp, synthetic construct
  source 1..888: mol_type = other DNA
gtgaaatatggcatttatttcgcatactgggaaaaatattgggatgttgatcaagttaaatatattaagaaagttaaagatctgggcttcgatattctggaagttagcggcgcactgctgaaaaataatacaagagaaagactgagagaaatcggcgaaatggcaaaagcagaaggcgttattctgacatgcggctatggcccgaatgcaaaagaggatctgggctcagcagatccggaagttagaaaaaatgcagttgcagtttatacagatctgctggaaaaaatgagaattatgaatattcatacatttggcggaggcatttattcatattggccggttgattattcaaaaccgattgataaagaaggcgattgggcaagatcagttgatggcgttagaacagttggcaaaattgcgggcgaatgcggcgttgattattgcctggaagttctgaatagatttgaaggctatctgattaatacatgcgaagaatgcatgaaatttgttgatgaagttggcgaaccggcagttaaagttatgctggatacatttcatatgaatattgaagaagatgatatgattaaagcaattagactggcgggcgataaactgggccattttcatgttggcgaaaataatagaaaactgccgggcaaaggcagatttccgtggagagaaattggacgcgcactgcaagatattggctatcaaaaaaatgttgttatggaaccgtttgttcaaatgggcggcgaagttggctcagatattaaagtttggagagatattagcggcggcgcaacgggcgaacaactggatagagatgcaaaagattcagttgcatttctgagatatgcatttgaaggcacgggcggcagactgtggtaa

SEQ ID NO: 28 - DNA, 864 bp, synthetic construct
  source 1..864: mol_type = other DNA
atgaaatatggcatctattatgcatattgggaaaaagaatggggcggagattttattccgtatgttgcaaaagttaaacaattaggctttgatgttcttgaagttgcatgcggagattttcataatcaaccggattcttatttacatgcactgcgcgaaacagcaaaagaaaatgacatcatcctgacgggcggctatggccctagagcagaacataatttagcatcaccggatccggcagttgttgaaaatgcacttgcattttatagcgaaatctttcgcaaaatggatattgcagacatacgctcaattggaggcgcactgtatagctattggccgatcgattataataaagaactggataaagatggcgatcttgaacgctcaattcaaggaatgcgcaaacttgcagatatagcaggacgctatggaattacacttaatatggaagttcttaatcgctttgaaggctatctgatcaatgatgttcgcgaaggactggcatatgttagagcagttgataaacctaatgttaaactgatgctggatacgtttcatatgaatatcgaagaagattctcttatcgaaccgatcctgctggcaggcaaagatctgggacatgttcatgttggcgaacctaatcgcaaacctcctcgcgaaggccgcatgccgtggaaagaaatcggcgttgcactgagagaaatcggctatgatggagcagttgttatggaaccgtttgttacgatgggaggacaagttggcaaagacataagaatctggcgcgatatttcacaaggagcaacggaaaatgatttagatagagatgcagcaaaaagcgttgcatttctgcgctcagcatttgaa

(19) United States
(12) Patent Application Publication
(10) Pub. No.: US 2025/0304942 A1
(43) Pub. Date: Oct. 2, 2025

(54) COMPOSITIONS AND METHODS FOR PRODUCING ALLULOSE

(71) Applicant: DANISCO US INC., Palo Alto, CA (US)

(72) Inventors: Zhongmei Tang, Shanghai (CN); Zhen Qian, Shanghai (CN); Sang-Kyu Lee, Mountain View, CA (US); Michael Pepsin, Castro Valley, CA (US)

(21) Appl. No.: 18/716,420

(22) PCT Filed: Dec. 14, 2022
(86) PCT No.: PCT/US2022/081510

§ 371 Date: Jun. 4, 2024

(30) Foreign Application Priority Data
Dec. 14, 2021 (WO) PCT/CN2021/137842

(51) Int. Cl. C12N 9/90; C12N 9/92; C12N 11/08; C12N 15/75; C12P 19/02; C12P 19/24

(52) U.S. Cl. CPC C12N 9/90; C12N 9/92; C12N 11/08; C12N 15/75; C12P 19/02; C12P 19/24

(57) ABSTRACT

Provided herein are compositions and methods relating to epimerase enzymes for converting fructose to allulose.

### CROSS-REFERENCE TO RELATED APPLICATION

This application is a U.S. National Stage Application under 35 U.S.C. § 371 of International Application No. PCT/US2022/081510, filed Dec. 14, 2022, which claims priority to International Application No. PCT/CN2021/137842, filed Dec. 14, 2021, the contents of which are hereby incorporated by reference in their entireties.

### REFERENCE TO A SEQUENCE LISTING

The contents of the electronic submission of the Sequence Listing, named “NB42012-US-PCT2_SL_ST25” was created on Dec. 23, 2024 and is 56,158 bytes in size, which is hereby incorporated by reference in its entirety.

### FIELD OF THE INVENTION

Provided herein are compositions and methods relating to epimerase enzymes for converting fructose to allulose.

### BACKGROUND

Allulose, also known as D-allulose and D-psicose, is a rare naturally occurring low calorie sugar having a sweetness profile similar to that of sucrose, making it a desirable alternative to higher calorie sweeteners, such as sucrose, fructose, and glucose. Allulose is a C-3 epimer of D-fructose, and may thus be produced, e.g., commercially, by conversion of D-fructose to allulose by enzymes such as epimerases.

Epimerases capable of converting D-fructose to allulose have been found to have a variety of properties, e.g., temperature, pH, and metal cofactor requirements, that can impact their enzymatic activity. Epimerases stable at high temperature and low pH with a reduced need for supplemented metal cofactors are of particular value for increasing the yield and quality of allulose during commercial production.

Thus, there is a need for epimerases capable of converting D-fructose to allulose at low pH and high temperature with a reduced need for added metal cofactors. The compositions and methods provided herein address these and other needs in the art.

### SUMMARY OF THE INVENTION

Provided herein are proteins including an amino acid sequence having at least 70% sequence identity to the sequence set forth by SEQ ID NO:2, SEQ ID NO:3, SEQ ID NO:4, SEQ ID NO: 5, SEQ ID NO:6, SEQ ID NO:7, SEQ ID NO:8, or SEQ ID NO:9, wherein the protein has epimerase activity. Provided herein are proteins including an amino acid sequence having at least 70% sequence identity to the sequence set forth by SEQ ID NO:19, SEQ ID NO:20, SEQ ID NO: 21, SEQ ID NO:22, or SEQ ID NO:23, wherein the protein has epimerase activity. In some embodiments, the amino acid sequence has at least 80%, 90%, 95%, 99%, or 100% sequence identity to the sequence set forth by SEQ ID NO:2, SEQ ID NO:3, SEQ ID NO:4, SEQ ID NO:5, SEQ ID NO:6, SEQ ID NO:7, SEQ ID NO:8, or SEQ ID NO:9. In some embodiments, the amino acid sequence has at least 80%, 90%, 95%, 99%, or 100% sequence identity to the sequence set forth by SEQ ID NO:19, SEQ ID NO:20, SEQ ID NO:21, SEQ ID NO:22, or SEQ ID NO:23. In some embodiments, the protein is immobilized on a matrix. In some embodiments, the matrix is a granule or an ion exchange resin.

In an aspect is provided a nucleic acid molecule comprising a nucleic acid sequence encoding a protein described herein. In some embodiments, the nucleic acid molecule includes a nucleic acid sequence that: i) encodes an amino acid sequence having at least 90%, 95%, 99%, or 100% sequence identity to the sequence set forth by SEQ ID NO:2, SEQ ID NO:3, SEQ ID NO:4, SEQ ID NO:5, SEQ ID NO:6, SEQ ID NO:7, SEQ ID NO:8, or SEQ ID NO:9; ii) has at least 70%, 80%, 90%, 95%, 99%, or 100% sequence identity to the sequence set forth by SEQ ID NO: 11, SEQ ID NO: 12, SEQ ID NO: 13, SEQ ID NO:14, SEQ ID NO: 15, SEQ ID NO: 16, SEQ ID NO: 17, or SEQ ID NO:18; or iii) hybridizes under stringent conditions to a nucleic acid sequence having a sequence complementary to the sequence set forth by SEQ ID NO: 11, SEQ ID NO: 12, SEQ ID NO: 13, SEQ ID NO: 14, SEQ ID NO: 15, SEQ ID NO: 16, SEQ ID NO:17, or SEQ ID NO: 18. In some embodiments, the nucleic acid molecule includes a nucleic acid sequence that: i) encodes an amino acid sequence having at least 90%, 95%, 99%, or 100% sequence identity to the sequence set forth by SEQ ID NO:19, SEQ ID NO:20, SEQ ID NO:21, SEQ ID NO:22, or SEQ ID NO:23; ii) has at least 70%, 80%, 90%, 95%, 99%, or 100% sequence identity to the sequence set forth by SEQ ID NO:24, SEQ ID NO:25, SEQ ID NO:26, SEQ ID NO:27, or SEQ ID NO: 28; or iii) hybridizes under stringent conditions to a nucleic acid sequence having a sequence complementary to the sequence set forth by SEQ ID NO:24, SEQ ID NO:25, SEQ ID NO: 26, SEQ ID NO:27, or SEQ ID NO:28. In some embodiments, the nucleic acid molecule includes a heterologous regulatory sequence. In some embodiments, the heterologous regulatory sequence is a promoter sequence.

In an aspect is provided a vector including a nucleic acid sequence described herein. In an aspect is provided a host cell including a nucleic acid molecule described herein or a vector described herein. In some embodiments, the host cell is a yeast, a bacterium, a mammalian cell, or a plant cell. In some embodiments, the host cell is a Bacillus spp. In some embodiments, the host cell is a Bacillus subtilis.

In an aspect is provided a cultured cell material including a protein described herein and/or a host cell described herein. In an aspect is provided allulose produced by a protein described herein.

In an aspect is provided a composition for producing allulose, including: i) a protein described herein; and ii) a substrate containing fructose. In some embodiments, the protein is immobilized on a matrix. In some embodiments, the substrate includes glucose. In some embodiments, the composition further includes a glucose isomerase immobilized on a matrix. In some embodiments, the protein and glucose isomerase are co-immobilized on the same matrix or immobilized on different matrixes. In some embodiments, the composition is contained in a reactor.

In an aspect is provide use of a protein described herein for producing allulose. In an aspect is provided a method of producing allulose, including contacting a protein described herein with a substrate comprising fructose. In some embodiments, the contacting occurs under conditions including a temperature in a range of about 50° C. to about 90° C. In some embodiments, the contacting occurs under conditions including a pH in a range of about 4.5 to about 8. In some embodiments, the contacting occurs under conditions where no metal cofactor is added or an amount of metal cofactor that is less than a metal cofactor concentration needed for epimerase activity is added. In some embodiments, the protein is soluble and contained in a reactor, and the contacting occurs by adding the substrate containing fructose to the reactor. In some embodiments, the protein is immobilized on a matrix contained in a reactor, and the contacting occurs by adding the substrate containing fructose to the reactor. In some embodiments, the substrate containing fructose is produced by: (i) contacting a substrate containing glucose with a glucose isomerase prior to contacting the protein; or (ii) contacting a substrate containing glucose with a glucose isomerase at the same time as contacting the protein. In some embodiments, the method includes purifying the produced allulose.

In an aspect is provided a kit including: (i) a protein described herein, a nucleic acid molecule described herein, a vector described herein, a host cell described herein, and/or a cell culture material described herein; and (ii) instructions for use.

Each of the aspects and embodiments described herein are capable of being used together, unless excluded either explicitly or clearly from the context of the embodiment or aspect.

### DETAILED DESCRIPTION

Provided herein are compositions and methods relating to epimerase enzymes, also referred to as epimerases, for converting D-fructose (D-fructose and fructose are used herein interchangeably) to allulose.

Allulose is a hexoketose monosaccharide sweetener, which is a C-3 epimer of D-fructose, that is rarely found in nature. Allulose has similar physical properties to those of sucrose, such as bulk, mouthfeel, browning capability, gelling, and freezing point, and its sweetness is estimated to be about 70% of the sweetness of sucrose. The energy value of allulose, however, is approximately 0.3% of that of sucrose. In addition to having low caloric value, allulose may have beneficial physiological effects, such as blood glucose suppression, reactive oxygen species scavenging, and neuroprotection among others. These properties have made allulose an attractive substitute for higher calorie sweeteners, e.g., sucrose, fructose, and glucose.

Since allulose is naturally present in only small quantities in certain foods, there exists a need for methods to efficiently and effectively produce allulose. The bio-conversion of D-fructose to D-allulose by epimerases, for example D-tagatose 3-epimerases (DT3E) and D-allulose 3-epimerases, is one such method of producing allulose. However, epimerases capable of performing this conversion, most of which have a bacterial origin, are known to have varying properties, such as temperature, pH, and metal cofactor requirements, that can impact enzymatic activity and efficiency. For example, most of the epimerases that have been identified as capable of performing this conversion show a dependence on manganese, cobalt, and/or magnesium as a cofactor to be active and optimal temperature and pH ranges for activity between 40° C. and 70° C. and 7.0 to 9.0 pH, respectively. For commercial production, it is preferable to use higher temperatures, e.g., about or greater than 50° C., to shift the thermodynamic equilibrium in favor of converting fructose to allulose, thereby increasing the ratio of allulose to fructose. It is also preferable to use an acidic pH, in particular at elevated temperatures, to reduce non-enzymatic browning of the sugars, e.g., via the Maillard reaction. The use of elevated temperatures and acidic pH in the production process provides additional advantages such as microbial control, for example by reducing microbial growth. In addition, for commercial production it is beneficial to use enzymes that do not require the addition (supplementation) or require a reduced amount of metal cofactors to be added for activity, as this would eliminate an additional step in the production process and/or reduce the cost of production. As described herein, D-allulose 3-epimerase homologs capable of converting D-fructose to allulose and having such desirable temperature, pH, and/or metal cofactor properties for commercial production were identified. See, Examples. The compositions and methods provided herein harness these surprising findings.

The conversion of D-fructose to D-allulose using the compositions, e.g., epimerases and/or immobilized compositions thereof, and methods described herein provide a means of producing allulose under preferable commercial conditions for epimerase activity and sugar stability. The use of the compositions and methods described herein may assist in diversifying the sweetener product portfolio associated with corn processing by adding a natural low calorie sweetener and bulking agent to the traditional sweeteners derived from corn starch (e.g., corn syrup, high fructose corn syrup (HFCS), glucose, and fructose).

The headings provided herein are not limitations of the various aspects or embodiments of this disclosure which can be had by reference to the specification as a whole. The section headings used herein are for organizational purposes only and are not to be construed as limiting the subject matter described. The reader will appreciate that statements made in one section may apply to other sections. Any terms defined may be more fully defined by reference to the specification as a whole.

All publications, including patent documents, scientific articles, and databases, referred to in this application are incorporated by reference in their entirety for all purposes to the same extent as if each individual publication were individually incorporated by reference. Nothing herein is to be construed as an admission that such publications constitute prior art to the claims appended hereto. If a definition set forth herein is contrary to or otherwise inconsistent with a definition set forth in the patents, applications, published applications, and other publications that are herein incorporated by reference, the definition set forth herein prevails over the definition that is incorporated herein by reference.

### Definitions

Definitions of terms may appear throughout the specification. It is to be understood that this disclosure is not limited to particular embodiments described, as such may, of course, vary. It is also to be understood that the terminology used herein is for the purpose of describing particular embodiments only and is not intended to be limiting.

It must be noted that as used herein and in the appended claims, the singular forms “a,” “an,” and “the” include plural referents unless the context clearly dictates otherwise. For example, “a” or “an” include “at least one” and “one or more.”

The terms “comprising”, “comprises,” and “comprised of” as used herein are synonymous with “including,” “includes,” “containing,” “contains,” and grammatical variants thereof, and are inclusive or open-ended and do not exclude additional, non-recited members, elements, or method steps. The terms “comprising,” “comprises,” “comprised of,” “including,” “includes,” “containing,” “contains,” and grammatical variants thereof also include the term “consisting of”.

Unless defined otherwise, all technical and scientific terms used herein have the same meaning as commonly understood by one of ordinary skill in the art.

The term “starch” refers to any material comprised of the complex polysaccharide carbohydrates of plants, comprised of amylose and amylopectin with the formula (C₆H₁₀O₅)x, wherein X can be any number. The term includes plant-based materials such as grains, cereal, grasses, tubers and roots, and more specifically materials obtained from wheat, barley, corn, rye, rice, sorghum, brans, cassava, millet, milo, potato, sweet potato, and tapioca. The term “starch” includes granular starch. The term “granular starch” refers to raw, i.e., uncooked starch, e.g., starch that has not been subject to gelatinization.

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

Reference to the wild-type polypeptide is understood to include the mature form of the polypeptide. A “mature” polypeptide or variant, thereof, is one in which a signal sequence is absent, for example, cleaved from an immature form of the polypeptide during or following expression of the polypeptide.

The term “variant,” with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. A variant may include two or more mutations, e.g., 2, 3, 4, 5, 6, 7, 8, 9, 10, or more, substitutions, deletions, and/or insertions compared to the wild-type, parental, or reference polypeptide or polynucleotide. The identity of the wild-type, parental, or reference polypeptide or polynucleotide will be apparent from context.

In the case of the epimerases described herein, “activity” refers to epimerase activity, which can be measured as described herein. It should be appreciated that epimerases operate bidirectionally as an equilibrium conversion reaction to interconvert fructose to allulose. In some embodiments, the activity includes or is the conversion of fructose to allulose. In some embodiments, the activity includes or is the conversion of allulose to fructose. Estimates of activity may be determined by assays designed to assess fructose formation from allulose, e.g., by colorimetric assay, and/or allulose formation from fructose, e.g., by high-performance liquid chromatography (HPLC). In some embodiments, the activity is referred to as a residual activity. As used herein, “residual activity” includes or is the activity of an epimerase following a challenge, e.g., elevated temperature challenge and/or pH challenge, compared to the activity of an unchallenged epimerase, which serves as a baseline for comparison, or is epimerase activity determined in a specific state, e.g., an immobilized state, compared to the activity of an epimerase in a different state (e.g., solubilized), which serves as a baseline. Residual activity may be expressed as a percentage or fraction of the baseline activity (e.g., baseline activity is equal to 100% or 1). Methods for determining activity of an enzyme are various and known in the art.

The term “recombinant,” when used in reference to a subject cell, nucleic acid, protein or vector, indicates that the subject has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding an epimerase may be referred to as a recombinant vector.

The terms “recovered,” “isolated,” and “separated,” refer to a compound, protein (polypeptide), cell, nucleic acid, amino acid, or other specified material or component that is removed from at least one other material or component. In some embodiments, the at least one other material or component is at least one other material or component with which the compound, protein (polypeptide), cell, nucleic acid, amino acid, or other specified material or component is naturally associated as found in nature. In some embodiments, the at least one other material or component is at least one other material or component with which the compound, protein (polypeptide), cell, nucleic acid, amino acid, or other specified material or component is associated with under experimental or production conditions and/or systems. For example, an “isolated” polypeptide includes, but is not limited to, a polypeptide removed from a culture broth containing a heterologous host cell expressing the polypeptide.

The term “purified” refers to material (e.g., an isolated compound, polypeptide, polynucleotide, or other specified material or component) that is in a relatively pure state, e.g., at least about 90% pure, at least about 95% pure, at least about 98% pure, or at least about 99% pure.

The term “enriched” refers to material (e.g., an isolated compound, polypeptide, polynucleotide, or other specified material or component) that is in about 50% pure, at least about 60% pure, at least about 70% pure, or even at least about 80% pure.

As used herein, “derived from” encompasses “originated from,” “obtained from,” or “isolated from.”

The terms “thermal stability,” “thermostable,” and “thermostability,” with reference to an enzyme, refer to the ability of the enzyme to retain activity at elevated temperatures or after exposure to an elevated temperature. Methods for determining thermostability are various and known in the art. In some cases, thermostability of an enzyme, such as an epimerase enzyme, may be measured by its half-life (t1/2) given in minutes, hours, or days, during which half the enzyme activity is lost under defined conditions. The half-life may be calculated by measuring residual epimerase activity following exposure to an elevated temperature. In some cases, thermostability is determined by measuring epimerase activity following exposure to an elevated temperature and comparing the measured activity against a baseline activity, where the baseline activity is measured from an epimerase that was not exposed to an elevated temperature. The value resulting from the comparison may be referred to as a residual activity.

A “pH range,” with reference to an enzyme, refers to the range of pH values under which the enzyme exhibits activity. The pH range where an enzyme demonstrates activity may be referred to as the “pH activity profile” of the enzyme. The terms “pH stable” and “pH stability,” with reference to an enzyme, relate to the ability of the enzyme to retain activity at a pH or after exposure to a pH. Methods for determining a pH profile and pH stability of an enzyme are various and known in the art. In some cases, the pH profile of an enzyme is determined by measuring the activity of the epimerase across a range of pHs. In this case, the minimum and maximum activity levels may be determined to produce a dose response curve or standard curve. In some cases, pH stability is determined by measuring epimerase activity following exposure to a pH and comparing the measured activity against a baseline activity, where the baseline activity is measured from an epimerase that was not exposed to the pH. The value resulting from the comparison may be referred to as a residual activity.

The term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “nucleic acid” encompasses DNA, RNA, heteroduplexes, and synthetic molecules capable of encoding a polypeptide. Nucleic acids may be single stranded or double stranded, and may contain chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

“Hybridization” refers to the process by which one strand of nucleic acid forms a duplex with, i.e., base pairs with, a complementary strand, as occurs during blot hybridization techniques and PCR techniques. Stringent hybridization conditions are exemplified by hybridization under the following conditions: 65° C. and 0.1×SSC (where 1×SSC=0.15 M NaCl, 0.015 M Na₃ citrate, pH 7.0). Hybridized, duplex nucleic acids are characterized by a melting temperature (Tm), where one half of the hybridized nucleic acids are unpaired with the complementary strand. Mismatched nucleotides within the duplex lower the Tm.

The terms “transformed,” “stably transformed,” and “transgenic,” used with reference to a cell means that the cell contains a non-native (e.g., heterologous) nucleic acid sequence integrated into its genome or carried as an episome that is maintained through multiple generations.

The term “introduced” in the context of inserting a nucleic acid sequence into a cell, encompasses, but is not limited to, “transfection”, “transformation” and “transduction,” as known in the art. Exemplary methods for introducing polynucleotides or polypeptides by transformation into a host cell, include, but are not limited to, microinjection, electroporation, stable transformation methods, transient transformation methods (such as induced competence using chemical (e.g. divalent cations such as CaCl₂)), mechanical (electroporation) means, or methods such as those described in published international applications WO 2018/114983 and WO 2010/149721, which are incorporated herein by reference in their entireties), ballistic particle acceleration (particle bombardment), direct gene transfer, viral-mediated introduction, cell-penetrating peptides, or mesoporous silica nanoparticle (MSN)-mediated direct protein delivery. Introducing a nucleic acid, construct, plasmid, or vector into a host cell may be carried out by conjugation, which is a specific method of natural DNA exchange requiring physical cell-to-cell contact. Introducing a nucleic acid, construct, plasmid, or vector into a host cell may be carried out by transduction, which is the introduction of DNA via a virus (e.g., phage) infection which is also a natural method of DNA exchange. Generally, such methods involve incorporating a polynucleotide within a viral DNA or RNA molecule.

A “host cell” is an organism into which an expression vector, phage, virus, or other nucleic acid sequence including a polynucleotide encoding a polypeptide of interest (e.g., an epimerase) has been introduced. Exemplary host cells are microorganism cells (e.g., bacteria, filamentous fungi, and yeast), mammalian cells, and plant cells capable of expressing the polypeptide of interest. The term “host cell” includes protoplasts created from cells.

The term “heterologous” with reference to a polynucleotide or protein refers to a polynucleotide or protein that does not naturally occur in a host cell.

The term “endogenous” with reference to a polynucleotide or protein refers to a polynucleotide or protein that occurs naturally in the host cell.

The term “expression” refers to the process by which a polypeptide is produced based on a nucleic acid sequence. The process includes both transcription and translation.

A “selective marker” or “selectable marker” refers to a gene capable of being expressed in a host to facilitate selection of host cells carrying the gene. Examples of selectable markers include but are not limited to antimicrobials (e.g., hygromycin, bleomycin, or chloramphenicol) and/or genes that confer a metabolic advantage, such as a nutritional advantage on the host cell.

A “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

A “signal sequence” is a sequence of amino acids attached to the N-terminal portion of a protein, which facilitates the secretion of the protein outside the cell. The mature form of an extracellular protein lacks the signal sequence, which is cleaved off during the secretion process.

The term “specific activity” refers to the number of moles of substrate that can be converted to product by an enzyme or enzyme preparation per unit time under specific conditions. Specific activity is generally expressed as units (U)/mg of protein.

“A cultured cell material comprising an epimerase” or similar language, refers to a cell lysate or supernatant (including media) that includes an epimerase as a component. The cell material may be from a heterologous host cell that is grown in culture for the purpose of producing the epimerase.

“Percent sequence identity” means that a particular sequence has at least a certain percentage of amino acid residues or nucleotides identical to those in a specified reference sequence, when aligned using e.g., the CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF.

Deletions are counted as non-identical residues, compared to a reference sequence. Deletions occurring at either termini are included.

The term “degree of polymerization” (DP) refers to the number (n) of anhydro-glucopyranose units in a given saccharide. Examples of DP1 are the monosaccharides glucose and fructose. Examples of DP2 are the disaccharides maltose and sucrose. The term “DE,” or “dextrose equivalent,” is defined as the percentage of reducing sugar, i.e., D-glucose, as a fraction of total carbohydrate in a syrup.

The term “dry solids content” (ds) refers to the total solids of a slurry in a dry weight percent basis. The term “slurry” refers to an aqueous mixture containing insoluble solids.

The phrase “simultaneous saccharification and fermentation (SSF)” refers to a process in the production of biochemicals in which a microbial organism, such as an ethanologenic microorganism, and at least one enzyme, such as an amylase, are present during the same process step. SSF includes the contemporaneous hydrolysis of starch substrates (granular, liquefied, or solubilized) to saccharides, including glucose, and the fermentation of the saccharides into alcohol or other biochemical or biomaterial in the same reactor vessel.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

Numerical values and ranges may be presented herein with the numerical value being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context. All values and ranges implicitly include the term “about” unless the context clearly dictates otherwise.

### I. EPIMERASES

Provided herein are epimerases and compositions containing epimerases useful for converting D-fructose to D-allulose. The epimerases described herein have functional properties that allow the enzymes to function under preferable conditions, e.g., stable sugar and equilibrium conversion conditions, for converting D-fructose to allulose. In addition, as further described in Section II, the epimerases provided herein can be used to produce allulose from output streams of existing procedures for producing fructose from starch.

In some aspects, the epimerases provided herein are D-allulose 3-epimerase homologs found in microorganisms, e.g., bacteria, that have an increased thermal stability, an increased pH stability or activity, and/or do not require or require less added metal cofactor, such as magnesium (Mg²⁺), compared to other D-allulose 3-epimerase homologs. See, e.g., Examples. Methods of determining thermostability, pH stability and activity, and metal cofactor requirements are known in the art and are also described in the Examples below (see, Section V).

The epimerases described herein may have thermal stability at elevated temperatures. In some embodiments, the epimerases described herein exhibit epimerase activity (converting fructose to allulose) at temperatures where the equilibrium is shifted to produce higher allulose conversion yield. In some embodiments, the epimerase is thermostable at a temperature of at least 40° C. In some embodiments, the epimerase is thermostable at a temperature of at least 50° C. In some embodiments, the epimerase is thermostable at temperatures in the range of about 40° C. to about 90° C. In some embodiments, the epimerase is thermostable at temperatures in the range of about 50° C. to about 90° C. In some embodiments, the epimerase is thermostable at temperatures in the range of about 50° C. to about 85° C. In some embodiments, the epimerase is thermostable at temperatures in the range of about 50° C. to about 80° C. In some embodiments, the epimerase is thermostable at temperatures in the range of about 50° C. to about 75° C. In some embodiments, the epimerase is thermostable at temperatures in the range of about 50° C. to about 70° C. In some embodiments, the epimerase is thermostable at temperatures in the range of about 60° C. to about 70° C. In some embodiments, the epimerase is thermostable at a temperature of about 60° C. In some embodiments, the epimerase is thermostable at a temperature of about 70° C. In some embodiments, the epimerase is thermostable at a temperature of about 80° C. In some embodiments, the epimerase is thermostable at a temperature of about 85° C. In some embodiments, the epimerase is thermostable at a temperature of about 90° C. In some embodiments, at a temperature in the range of about 50° C. to about 90° C., the epimerase retains at least 50%, 60%, 70%, 75%, 80%, 85%, 90%, 91%, 92%, 93%, 94%, 95%, 96%, 97%, 98%, 99%, or 100% of a baseline activity. In some embodiments, at a temperature in the range of about 50° C. to about 85° C., the epimerase retains at least 50%, 60%, 70%, 75%, 80%, 85%, 90%, 91%, 92%, 93%, 94%, 95%, 96%, 97%, 98%, 99%, or 100% of a baseline activity. In some embodiments, at a temperature in the range of about 50° C. to about 80° C., the epimerase retains at least 50%, 60%, 70%, 75%, 80%, 85%, 90%, 91%, 92%, 93%, 94%, 95%, 96%, 97%, 98%, 99%, or 100% of a baseline activity. In some embodiments, at a temperature in the range of about 60° C. to about 80° C., the epimerase retains at least 50%, 60%, 70%, 75%, 80%, 85%, 90%, 91%, 92%, 93%, 94%, 95%, 96%, 97%, 98%, 99%, or 100% of a baseline activity. In some embodiments, at a temperature in the range of about 60° C. to about 75° C., the epimerase retains at least 75%, 80%, 85%, 90%, 91%, 92%, 93%, 94%, 95%, 96%, 97%, 98%, 99%, or 100% of a baseline activity. In some embodiments, at a temperature in the range of about 60° C. to about 70° C., the epimerase retains at least 75%, 80%, 85%, 90%, 91%, 92%, 93%, 94%, 95%, 96%, 97%, 98%, 99%, or 100% of a baseline activity. In some embodiments, at a temperature in the range of about 60° C. to about 70° C., the epimerase retains about 50% to 100% of a baseline activity. In some embodiments, at a temperature in the range of about 60° C. to about 70° C., the epimerase retains about 75% to 100% of a baseline activity. In some embodiments, at a temperature in the range of about 60° C. to about 70° C., the epimerase retains about 80% to 100% of a baseline activity. In some embodiments, at a temperature in the range of about 60° C. to about 70° C., the epimerase retains about 90% to 100% of a baseline activity. The baseline activity may be an activity determined for an epimerase that was not exposed to a temperature (e.g., elevated temperature) as described in this paragraph. In some embodiments, the baseline activity is an activity determined for an epimerase at a temperature of about 50° C. In some embodiments, the retained activity is sufficient for converting fructose to allulose.

Thermostability may be determined as a residual activity measured after an epimerase has been exposed to (e.g., incubated at) an elevated temperature, for example for a particular duration. In some embodiments, the epimerase retains residual activity following incubation at a temperature in the range of about 60° C. to about 70° C. for a duration of between about 5 min to about 120 min. In some embodiments, the residual activity retained following incubation is at least 50%, 60%, 70%, 75%, 80, %, 85%, 90%, 91%, 92%, 93%, 94%, 95%, 96%, 97%, 98%, 99%, or 100% of a baseline activity. In some embodiments, the residual activity retained following incubation is at least or about 50% of a baseline activity. In some embodiments, the residual activity retained following incubation is at least or about 60% of a baseline activity. In some embodiments, the residual activity retained following incubation is at least or about 70% of a baseline activity. In some embodiments, the residual activity retained following incubation is at least or about 80% of a baseline activity. In some embodiments, the residual activity retained following incubation is at least or about 90% of a baseline activity. In some embodiments, the epimerase retains about 50% to 100% of a baseline activity. In some embodiments, the epimerase retains about 75% to 100% of a baseline activity. In some embodiments, the epimerase retains about 80% to 100% of a baseline activity. In some embodiments, the epimerase retains about 90% to 100% of a baseline activity. The baseline activity may be an activity determined for an epimerase that was not subjected to an elevated temperature, e.g., a temperature as described above and in the preceding paragraph. In some embodiments, the baseline activity is an activity determined for an epimerase at a temperature of about 50° C.

The epimerases for use described herein may have desirable pH stability. For example, the epimerases described herein may have pH stability in a range of pHs where fructose and/or allulose are stable. In some embodiments, the epimerase is pH stable in a pH range that reduces non-enzymatic browning of the sugars via the Maillard reaction. For example, the pH may be in a range where the Maillard reaction proceeds at a slower rate compared to a neutral or basic pH such as 7, 7.5, 8.5, 9, 9.5, or 10 pH. In some embodiments, the epimerase has a pH activity profile that is or overlaps with the pH range that reduces non-enzymatic browning of the sugars via the Maillard reaction. In some embodiments, the epimerase is pH stable in a pH range that reduces non-enzymatic browning of the sugars via the Maillard reaction at a given temperature. In some embodiments, the epimerase has a pH activity profile that is or overlaps with the pH range that reduces non-enzymatic browning of the sugars via the Maillard reaction at a given temperature. In some embodiments, the pH range may be in a range that reduces the speed of the Maillard reaction at a specific temperature compared to a neutral or basic pH such as 7, 7.5, 8.5, 9, 9.5, or 10 pH, at the same temperature. In some embodiments, the epimerase is stable at a pH in the range of about 4 to about 10. In some embodiments, the epimerase is stable at a pH in the range of about 4.5 to about 10. In some embodiments, the epimerase is stable at a pH in the range of about 4.5 to about 9. In some embodiments, the epimerase is stable at a pH in the range of about 4.5 to about 8. In some embodiments, the epimerase is stable at a pH in the range of about 5 to about 8. In some embodiments, the epimerase is stable at a pH in the range of about 5 to about 7. In some embodiments, the epimerase is stable at a pH in the range of about 5.5 to about 6.5. The range of pHs where an epimerase is stable may also be referred to herein as a pH range. For example, the ranges of pHs described above may be referred to as a pH range.

In some embodiments, the epimerase retains a level of activity of at least 30%, 40%, 50%, 60%, 70%, 75%, 80%, 85%, 90%, 91%, 92%, 93%, 94%, 95%, 96%, 97%, 98%, 99%, or 100% of a maximal activity level across the pH range. In some embodiments, the epimerase retains a level of activity of at least or about 25% of a maximal activity level across the pH range. In some embodiments, the epimerase retains a level of activity of at least or about 45% of a maximal activity level across the pH range. In some embodiments, the epimerase retains a level of activity of at least or about 50% of a maximal activity level across the pH range. In some embodiments, the epimerase retains a level of activity of at least or about 60% of a maximal activity level across the pH range. In some embodiments, the epimerase retains a level of activity of at least or about 70% of a maximal activity level across the pH range. In some embodiments, the epimerase retains a level of activity of at least or about 75% of a maximal activity level across the pH range. In some embodiments, the epimerase retains a level of activity in the range of about 50% to about 100% of a maximal activity level across the pH range. In some embodiments, the epimerase retains a level of activity in the range of about 75% to about 100% of a maximal activity level across the pH range. In some embodiments, the epimerase retains a level of activity in the range of about 80% to about 100% of a maximal activity level across the pH range. In some embodiments, the epimerase retains a level of activity in the range of about 90% to about 100% of a maximal activity level across the pH range. In some embodiments, the pH range is a pH range described in the preceding paragraph. In some embodiments, the pH range is from about 4.5 to about 10. In some embodiments, the pH range is from about 4.5 to about 9. In some embodiments, the pH range is from about 4.5 to about 8. In some embodiments, the pH range is from about 4.5 to about 7.5. In some embodiments, the pH range is from about 4.5 to about 7. In some embodiments, the pH range is from about 4.5 to about 6.5. In some embodiments, the pH range is from about 4.5 to about 6. In some embodiments, the pH range is from about 5 to about 6.5. In some embodiments, the pH range is from about 5.5 to about 6.5. The maximal level of activity may be determined by measuring the activity of the epimerase across a range of pHs in order to find minimum and maximum activity levels, e.g., characterize a dose response curve or standard curve. In some embodiments, the retained activity is sufficient for converting fructose to allulose.

The epimerases for use described herein may have a reduced or no need for supplemented metal cofactors for activity. In some embodiments, the epimerases described herein retain an activity observed in the presence of a metal cofactor, e.g., when a metal cofactor is intentionally added, when no or a reduced amount of metal cofactor is added. Thus, in some embodiments, the epimerases described herein do not require or require a reduced amount of metal cofactor to be supplemented in order for the epimerase to be active. In some embodiments, the epimerases described herein use existing metal cofactors present in the production process to be active. In some embodiments, the production process is a process for producing a substrate containing fructose (see, e.g., Section II-A). In some embodiments, the epimerase retains at least 50%, 60%, 70%, 75%, 80%, 85%, 90%, 91%, 92%, 93%, 94%, 95%, 96%, 97%, 98%, 99%, or 100% activity of a baseline activity in the absence of added metal cofactor. In some embodiments, the epimerase retains at least or about 50% activity of a baseline activity in the absence of added metal cofactor. In some embodiments, the epimerase retains at least or about 60% activity in the absence of added metal cofactor. In some embodiments, the epimerase retains at least or about 70% activity of a baseline activity in the absence of added metal cofactor. In some embodiments, the epimerase retains at least or about 75% activity of a baseline activity in the absence of added metal cofactor. In some embodiments, the epimerase retains at least or about 80% activity of a baseline activity in the absence of added metal cofactor. In some embodiments, the epimerase retains at least or about 90% activity of a baseline activity in the absence of added metal cofactor. In some embodiments, the epimerase retains about 50% to 100% activity of a baseline activity in the absence of added metal cofactor. In some embodiments, the epimerase retains about 75% to 100% activity of a baseline activity in the absence of added metal cofactor. In some embodiments, the epimerase retains about 80% to 100% activity of a baseline activity in the absence of added metal cofactor. In some embodiments, the epimerase retains about 90% to 100% activity of a baseline activity in the absence of added metal cofactor. In some embodiments, the baseline activity is an activity measured in the presence of a metal cofactor in a concentration range of about 0.1 to about 2 mM, about 0.1 to about 1 mM, about 0.1 to about 0.5 mM, about 0.1 to about 0.25 mM, or about 0.1 to about 0.15 mM. In some embodiments, the epimerase retains a percentage of activity as described in this paragraph and/or herein when an amount of metal cofactor that is less than the concentration of metal cofactor present for the baseline activity is added. In some embodiments, a reduced amount of metal cofactor is a concentration less than the concentration of metal cofactor present for the baseline activity. In some embodiments, the reduced amount is at least or about 20, 30, 40, 50 60, 70, 80, 90, 95, or 99% less than the concentration of metal cofactor present for the baseline activity. In some embodiments, the reduced amount is in a range of about 20% to about 90% less than the concentration of metal cofactor present for the baseline activity. In some embodiments, the reduced amount is in a range of about 30% to about 90% less than the concentration of metal cofactor present for the baseline activity. In some embodiments, the reduced amount is in a range of about 40% to about 90% less than the concentration of metal cofactor present for the baseline activity. In some embodiments, the reduced amount is in a range of about 50% to about 90% less than the concentration of metal cofactor present for the baseline activity. In some embodiments, the retained activity is sufficient for converting fructose to allulose. In some embodiments, the metal cofactor is an ion. In some embodiments, the metal cofactor is magnesium. In some embodiments, the metal cofactor is in the form of a salt. In some embodiments, the metal cofactor is a magnesium salt.

The epimerases for use described herein may have any one or more of a thermal stability, a pH stability, and/or reduced or no requirement for an added metal cofactor as described above and herein. In some embodiments, the epimerase is thermal stable, pH stable, and has a reduced or no dependence on added metal cofactors. In some embodiments, the epimerase is thermal stable. In some embodiments, the epimerase is pH stable. In some embodiments, the epimerase has reduced or no requirement for an added metal cofactor.

The epimerases described herein having features as described above and herein may be described as proteins, nucleic acid molecules, as part of vectors, or in compositions. Also provided herein are methods of producing such epimerases.

### A. Proteins

In an aspect is provided epimerase enzymes that are proteins having epimerase activity and are capable of converting D-fructose to allulose. In some embodiments, the protein is a D-allulose 3-epimerase. In some embodiments, the epimerase has one or more of the thermal stability, pH stability, or metal cofactor requirement attributes described herein.

In some embodiments, the protein, i.e., epimerase protein, includes or is an amino acid sequence having at least 70%, 80%, 90%, 91%, 92%, 93%, 94%, 95%, 96%, 97%, 98%, 99%, or 100% sequence identity to the sequence set forth by SEQ ID NO:2, SEQ ID NO:3, SEQ ID NO:4, SEQ ID NO:5, SEQ ID NO:6, SEQ ID NO:7, SEQ ID NO:8, or SEQ ID NO:9, where the protein has epimerase activity. In some embodiments, the protein includes or is an amino acid sequence having at least 70% sequence identity to the sequence set forth by SEQ ID NO:2, SEQ ID NO:3, SEQ ID NO:4, SEQ ID NO:5, SEQ ID NO:6, SEQ ID NO:7, SEQ ID NO:8, or SEQ ID NO:9, and where the protein has epimerase activity. In some embodiments, the protein includes or is an amino acid sequence having at least 80% sequence identity to the sequence set forth by SEQ ID NO: 2, SEQ ID NO:3, SEQ ID NO:4, SEQ ID NO:5, SEQ ID NO:6, SEQ ID NO:7, SEQ ID NO:8, or SEQ ID NO:9, and where the protein has epimerase activity. In some embodiments, the protein includes or is an amino acid sequence having at least 90% sequence identity to the sequence set forth by SEQ ID NO:2, SEQ ID NO:3, SEQ ID NO:4, SEQ ID NO:5, SEQ ID NO:6, SEQ ID NO: 7, SEQ ID NO:8, or SEQ ID NO:9, and where the protein has epimerase activity.

In some embodiments, the protein, i.e., epimerase protein, includes or is an amino acid sequence having at least 70%, 80%, 90%, 91%, 92%, 93%, 94%, 95%, 96%, 97%, 98%, 99%, or 100% sequence identity to the sequence set forth by SEQ ID NO:19, SEQ ID NO:20, SEQ ID NO: 21, SEQ ID NO:22, or SEQ ID NO:23, where the protein has epimerase activity. In some embodiments, the protein includes or is an amino acid sequence having at least 70% sequence identity to the sequence set forth by SEQ ID NO: 19, SEQ ID NO:20, SEQ ID NO:21, SEQ ID NO: 22, or SEQ ID NO:23, and where the protein has epimerase activity. In some embodiments, the protein includes or is an amino acid sequence having at least 80% sequence identity to the sequence set forth by SEQ ID NO: 19, SEQ ID NO:20, SEQ ID NO:21, SEQ ID NO:22, or SEQ ID NO: 23, and where the protein has epimerase activity. In some embodiments, the protein includes or is an amino acid sequence having at least 90% sequence identity to the sequence set forth by SEQ ID NO: 19, SEQ ID NO:20, SEQ ID NO:21, SEQ ID NO:22, or SEQ ID NO:23, and where the protein has epimerase activity.

In some embodiments, the protein includes or is an amino acid sequence having at least 70%, 75%, 80%, 85%, 90%, 91%, 92%, 93%, 94%, 95%, 96%, 97%, 98%, or 99% sequence identity to the sequence set forth by SEQ ID NO:2, and the protein has epimerase activity. In some embodiments, the protein includes or is an amino acid sequence having at least 80%, 85%, 90%, 91%, 92%, 93%, 94%, 95%, 96%, 97%, 98%, or 99% sequence identity to the sequence set forth by SEQ ID NO:2. In some embodiments, the protein includes or is an amino acid sequence having at least 90%, 91%, 92%, 93%, 94%, 95%, 96%, 97%, 98%, or 99% sequence identity to the sequence set forth by SEQ ID NO:2. In some embodiments, the protein includes or is the amino acid sequence set forth by SEQ ID NO:2.

In some embodiments, the protein includes or is an amino acid sequence having at least 70%, 75%, 80%, 85%, 90%, 91%, 92%, 93%, 94%, 95%, 96%, 97%, 98%, or 99% sequence identity to the sequence set forth by SEQ ID NO:3, and the protein has epimerase activity. In some embodiments, the protein includes or is an amino acid sequence having at least 80%, 85%, 90%, 91%, 92%, 93%, 94%, 95%, 96%, 97%, 98%, or 99% sequence identity to the sequence set forth by SEQ ID NO:3. In some embodiments, the protein includes or is an amino acid sequence having at least 90%, 91%, 92%, 93%, 94%, 95%, 96%, 97%, 98%, or 99% sequence identity to the sequence set forth by SEQ ID NO:3. In some embodiments, the protein includes or is the amino acid sequence set forth by SEQ ID NO:3.

In some embodiments, the protein includes or is an amino acid sequence having at least 70%, 75%, 80%, 85%, 90%, 91%, 92%, 93%, 94%, 95%, 96%, 97%, 98%, or 99% sequence identity to the sequence set forth by SEQ ID NO:4, and the protein has epimerase activity. In some embodiments, the protein includes or is an amino acid sequence having at least 80%, 85%, 90%, 91%, 92%, 93%, 94%, 95%, 96%, 97%, 98%, or 99% sequence identity to the sequence set forth by SEQ ID NO:4. In some embodiments, the protein includes or is an amino acid sequence having at least 90%, 91%, 92%, 93%, 94%, 95%, 96%, 97%, 98%, or 99% sequence identity to the sequence set forth by SEQ ID NO:4. In some embodiments, the protein includes or is the amino acid sequence set forth by SEQ ID NO:4.

In some embodiments, the protein includes or is an amino acid sequence having at least 70%, 75%, 80%, 85%, 90%, 91%, 92%, 93%, 94%, 95%, 96%, 97%, 98%, or 99% sequence identity to the sequence set forth by SEQ ID NO:5, and the protein has epimerase activity. In some embodiments, the protein includes or is an amino acid sequence having at least 80%, 85%, 90%, 91%, 92%, 93%, 94%, 95%, 96%, 97%, 98%, or 99% sequence identity to the sequence set forth by SEQ ID NO:5. In some embodiments, the protein includes or is an amino acid sequence having at least 90%, 91%, 92%, 93%, 94%, 95%, 96%, 97%, 98%, or 99% sequence identity to the sequence set forth by SEQ ID NO:5. In some embodiments, the protein includes or is the amino acid sequence set forth by SEQ ID NO:5.

In some embodiments, the protein includes or is an amino acid sequence having at least 70%, 75%, 80%, 85%, 90%, 91%, 92%, 93%, 94%, 95%, 96%, 97%, 98%, or 99% sequence identity to the sequence set forth by SEQ ID NO:6 and the protein has epimerase activity. In some embodiments, the protein includes or is an amino acid sequence having at least 80%, 85%, 90%, 91%, 92%, 93%, 94%, 95%, 96%, 97%, 98%, or 99% sequence identity to the sequence set forth by SEQ ID NO:6. In some embodiments, the protein includes or is an amino acid sequence having at least 90%, 91%, 92%, 93%, 94%, 95%, 96%, 97%, 98%, or 99% sequence identity to the sequence set forth by SEQ ID NO:6. In some embodiments, the protein includes or is the amino acid sequence set forth by SEQ ID NO:6.

In some embodiments, the protein includes or is an amino acid sequence having at least 70%, 75%, 80%, 85%, 90%, 91%, 92%, 93%, 94%, 95%, 96%, 97%, 98%, or 99% sequence identity to the sequence set forth by SEQ ID NO:7, and the protein has epimerase activity. In some embodiments, the protein includes or is an amino acid sequence having at least 80%, 85%, 90%, 91%, 92%, 93%, 94%, 95%, 96%, 97%, 98%, or 99% sequence identity to the sequence set forth by SEQ ID NO:7. In some embodiments, the protein includes or is an amino acid sequence having at least 90%, 91%, 92%, 93%, 94%, 95%, 96%, 97%, 98%, or 99% sequence identity to the sequence set forth by SEQ ID NO:7. In some embodiments, the protein includes or is the amino acid sequence set forth by SEQ ID NO:7.

In some embodiments, the protein includes or is an amino acid sequence having at least 70%, 75%, 80%, 85%, 90%, 91%, 92%, 93%, 94%, 95%, 96%, 97%, 98%, or 99% sequence identity to the sequence set forth by SEQ ID NO:8, and the protein has epimerase activity. In some embodiments, the protein includes or is an amino acid sequence having at least 80%, 85%, 90%, 91%, 92%, 93%, 94%, 95%, 96%, 97%, 98%, or 99% sequence identity to the sequence set forth by SEQ ID NO:8. In some embodiments, the protein includes or is an amino acid sequence having at least 90%, 91%, 92%, 93%, 94%, 95%, 96%, 97%, 98%, or 99% sequence identity to the sequence set forth by SEQ ID NO:8. In some embodiments, the protein includes or is the amino acid sequence set forth by SEQ ID NO:8.

In some embodiments, the protein includes or is an amino acid sequence having at least 70%, 75%, 80%, 85%, 90%, 91%, 92%, 93%, 94%, 95%, 96%, 97%, 98%, or 99% sequence identity to the sequence set forth by SEQ ID NO:9, and the protein has epimerase activity. In some embodiments, the protein includes or is an amino acid sequence having at least 80%, 85%, 90%, 91%, 92%, 93%, 94%, 95%, 96%, 97%, 98%, or 99% sequence identity to the sequence set forth by SEQ ID NO:9. In some embodiments, the protein includes or is an amino acid sequence having at least 90%, 91%, 92%, 93%, 94%, 95%, 96%, 97%, 98%, or 99% sequence identity to the sequence set forth by SEQ ID NO:9. In some embodiments, the protein includes or is the amino acid sequence set forth by SEQ ID NO:9.

In some embodiments, the protein includes or is an amino acid sequence having at least 70%, 75%, 80%, 85%, 90%, 91%, 92%, 93%, 94%, 95%, 96%, 97%, 98%, or 99% sequence identity to the sequence set forth by SEQ ID NO:19, and the protein has epimerase activity. In some embodiments, the protein includes or is an amino acid sequence having at least 80%, 85%, 90%, 91%, 92%, 93%, 94%, 95%, 96%, 97%, 98%, or 99% sequence identity to the sequence set forth by SEQ ID NO:19. In some embodiments, the protein includes or is an amino acid sequence having at least 90%, 91%, 92%, 93%, 94%, 95%, 96%, 97%, 98%, or 99% sequence identity to the sequence set forth by SEQ ID NO:19. In some embodiments, the protein includes or is the amino acid sequence set forth by SEQ ID NO:19.

In some embodiments, the protein includes or is an amino acid sequence having at least 70%, 75%, 80%, 85%, 90%, 91%, 92%, 93%, 94%, 95%, 96%, 97%, 98%, or 99% sequence identity to the sequence set forth by SEQ ID NO:20, and the protein has epimerase activity. In some embodiments, the protein includes or is an amino acid sequence having at least 80%, 85%, 90%, 91%, 92%, 93%, 94%, 95%, 96%, 97%, 98%, or 99% sequence identity to the sequence set forth by SEQ ID NO:20. In some embodiments, the protein includes or is an amino acid sequence having at least 90%, 91%, 92%, 93%, 94%, 95%, 96%, 97%, 98%, or 99% sequence identity to the sequence set forth by SEQ ID NO:20. In some embodiments, the protein includes or is the amino acid sequence set forth by SEQ ID NO:20.

In some embodiments, the protein includes or is an amino acid sequence having at least 70%, 75%, 80%, 85%, 90%, 91%, 92%, 93%, 94%, 95%, 96%, 97%, 98%, or 99% sequence identity to the sequence set forth by SEQ ID NO:21, and the protein has epimerase activity. In some embodiments, the protein includes or is an amino acid sequence having at least 80%, 85%, 90%, 91%, 92%, 93%, 94%, 95%, 96%, 97%, 98%, or 99% sequence identity to the sequence set forth by SEQ ID NO:21. In some embodiments, the protein includes or is an amino acid sequence having at least 90%, 91%, 92%, 93%, 94%, 95%, 96%, 97%, 98%, or 99% sequence identity to the sequence set forth by SEQ ID NO:21. In some embodiments, the protein includes or is the amino acid sequence set forth by SEQ ID NO:21.

In some embodiments, the protein includes or is an amino acid sequence having at least 70%, 75%, 80%, 85%, 90%, 91%, 92%, 93%, 94%, 95%, 96%, 97%, 98%, or 99% sequence identity to the sequence set forth by SEQ ID NO:22, and the protein has epimerase activity. In some embodiments, the protein includes or is an amino acid sequence having at least 80%, 85%, 90%, 91%, 92%, 93%, 94%, 95%, 96%, 97%, 98%, or 99% sequence identity to the sequence set forth by SEQ ID NO:22. In some embodiments, the protein includes or is an amino acid sequence having at least 90%, 91%, 92%, 93%, 94%, 95%, 96%, 97%, 98%, or 99% sequence identity to the sequence set forth by SEQ ID NO:22. In some embodiments, the protein includes or is the amino acid sequence set forth by SEQ ID NO:22.

In some embodiments, the protein includes or is an amino acid sequence having at least 70%, 75%, 80%, 85%, 90%, 91%, 92%, 93%, 94%, 95%, 96%, 97%, 98%, or 99% sequence identity to the sequence set forth by SEQ ID NO:23, and the protein has epimerase activity. In some embodiments, the protein includes or is an amino acid sequence having at least 80%, 85%, 90%, 91%, 92%, 93%, 94%, 95%, 96%, 97%, 98%, or 99% sequence identity to the sequence set forth by SEQ ID NO:23. In some embodiments, the protein includes or is an amino acid sequence having at least 90%, 91%, 92%, 93%, 94%, 95%, 96%, 97%, 98%, or 99% sequence identity to the sequence set forth by SEQ ID NO:23. In some embodiments, the protein includes or is the amino acid sequence set forth by SEQ ID NO:23.

In some embodiments, the protein (i.e., epimerase protein) may have any number of conservative amino acid substitutions, which are well recognized in the art. The present epimerases may be “precursor,” “immature,” or “full-length,” in which case they include a signal sequence, or “mature,” in which case they lack a signal sequence. Mature forms of the protein are generally the most useful. The present epimerases may also be truncated to remove the N or C-termini, or extended to include additional N or C-terminal residues, so long as the resulting protein retains activity.

### B. Nucleic Acid Molecules

In another aspect, nucleic acid molecules that are or contain a nucleic acid sequence encoding an epimerase are provided. The nucleic acid sequence may encode a particular epimerase described herein, or an epimerase having a specified degree of amino acid sequence identity to the particular epimerase.

In some embodiments, the nucleic acid molecule is or contains a nucleic acid sequence encoding an amino acid sequence having at least 70%, 80%, 90%, 95%, 99%, or 100% sequence identity to the sequence set forth by of SEQ ID NO:2, SEQ ID NO:3, SEQ ID NO:4, SEQ ID NO: 5, SEQ ID NO:6, SEQ ID NO:7, SEQ ID NO:8, or SEQ ID NO:9. In some embodiments, the nucleic acid molecule is or contains a nucleic acid sequence encoding an amino acid sequence having at least 70%, 80%, 90%, 95%, 99%, or 100% sequence identity to the sequence set forth by of SEQ ID NO: 19, SEQ ID NO:20, SEQ ID NO:21, SEQ ID NO:22, or SEQ ID NO:23. In some embodiments, the nucleic acid molecule is or contains a nucleic acid sequence encoding an amino acid sequence having at least 85%, 86%, 87%, 88%, 89%, 90%, 91%, 92%, 93%, 94%, 95%, 96%, 97%, 98%, 99%, or 100% sequence identity to the sequence set forth by SEQ ID NO:2, SEQ ID NO:3, SEQ ID NO:4, SEQ ID NO:5, SEQ ID NO:6, SEQ ID NO:7, SEQ ID NO:8, or SEQ ID NO:9. In some embodiments, the nucleic acid molecule is or contains a nucleic acid sequence encoding an amino acid sequence having at least 85%, 86%, 87%, 88%, 89%, 90%, 91%, 92%, 93%, 94%, 95%, 96%, 97%, 98%, 99%, or 100% sequence identity to the sequence set forth by SEQ ID NO:19, SEQ ID NO:20, SEQ ID NO:21, SEQ ID NO:22, or SEQ ID NO:23. In some embodiments, the nucleic acid molecule is or contains a nucleic acid sequence encoding an amino acid sequence having at least 90%, 91%, 92%, 93%, 94%, 95%, 96%, 97%, 98%, 99%, or 100% sequence identity to the sequence set forth by of SEQ ID NO:2, SEQ ID NO:3, SEQ ID NO: 4, SEQ ID NO:5, SEQ ID NO:6, SEQ ID NO:7, SEQ ID NO:8, or SEQ ID NO:9. In some embodiments, the nucleic acid molecule is or contains a nucleic acid sequence encoding an amino acid sequence having at least 90%, 91%, 92%, 93%, 94%, 95%, 96%, 97%, 98%, 99%, or 100% sequence identity to the sequence set forth by of SEQ ID NO:19, SEQ ID NO:20, SEQ ID NO:21, SEQ ID NO:22, or SEQ ID NO:23. In some embodiments, the nucleic acid molecule is or contains a nucleic acid sequence encoding the amino acid sequence set forth by of SEQ ID NO:2 or a nucleic acid sequence encoding an amino acid sequence having at least 90%, 91%, 92%, 93%, 94%, 95%, 96%, 97%, 98%, 99%, or 100% sequence identity to the sequence set forth by of SEQ ID NO: 2. In some embodiments, the nucleic acid molecule is or contains a nucleic acid sequence encoding the amino acid sequence set forth by of SEQ ID NO:3 or a nucleic acid sequence encoding an amino acid sequence having at least 90%, 91%, 92%, 93%, 94%, 95%, 96%, 97%, 98%, 99%, or 100% sequence identity to the sequence set forth by of SEQ ID NO:3. In some embodiments, the nucleic acid molecule is or contains a nucleic acid sequence encoding the amino acid sequence set forth by of SEQ ID NO:4 or a nucleic acid sequence encoding an amino acid sequence having at least 90%, 91%, 92%, 93%, 94%, 95%, 96%, 97%, 98%, 99%, or 100% sequence identity to the sequence set forth by of SEQ ID NO:4. In some embodiments, the nucleic acid molecule is or contains a nucleic acid sequence encoding the amino acid sequence set forth by of SEQ ID NO:5 or a nucleic acid sequence encoding an amino acid sequence having at least 90%, 91%, 92%, 93%, 94%, 95%, 96%, 97%, 98%, 99%, or 100% sequence identity to the sequence set forth by of SEQ ID NO:5. In some embodiments, the nucleic acid molecule is or contains a nucleic acid sequence encoding the amino acid sequence set forth by of SEQ ID NO:6 or a nucleic acid sequence encoding an amino acid sequence having at least 90%, 91%, 92%, 93%, 94%, 95%, 96%, 97%, 98%, 99%, or 100% sequence identity to the sequence set forth by of SEQ ID NO: 6. In some embodiments, the nucleic acid molecule is or contains a nucleic acid sequence encoding the amino acid sequence set forth by of SEQ ID NO:7 or a nucleic acid sequence encoding an amino acid sequence having at least 90%, 91%, 92%, 93%, 94%, 95%, 96%, 97%, 98%, 99%, or 100% sequence identity to the sequence set forth by of SEQ ID NO:7. In some embodiments, the nucleic acid molecule is or contains a nucleic acid sequence encoding the amino acid sequence set forth by of SEQ ID NO:8 or a nucleic acid sequence encoding an amino acid sequence having at least 90%, 91%, 92%, 93%, 94%, 95%, 96%, 97%, 98%, 99%, or 100% sequence identity to the sequence set forth by of SEQ ID NO:8. In some embodiments, the nucleic acid molecule is or contains a nucleic acid sequence encoding the amino acid sequence set forth by of SEQ ID NO:9 or a nucleic acid sequence encoding an amino acid sequence having at least 90%, 91%, 92%, 93%, 94%, 95%, 96%, 97%, 98%, 99%, or 100% sequence identity to the sequence set forth by of SEQ ID NO:9. In some embodiments, the nucleic acid molecule is or contains a nucleic acid sequence encoding the amino acid sequence set forth by of SEQ ID NO: 19 or a nucleic acid sequence encoding an amino acid sequence having at least 90%, 91%, 92%, 93%, 94%, 95%, 96%, 97%, 98%, 99%, or 100% sequence identity to the sequence set forth by of SEQ ID NO: 19. In some embodiments, the nucleic acid molecule is or contains a nucleic acid sequence encoding the amino acid sequence set forth by of SEQ ID NO:20 or a nucleic acid sequence encoding an amino acid sequence having at least 90%, 91%, 92%, 93%, 94%, 95%, 96%, 97%, 98%, 99%, or 100% sequence identity to the sequence set forth by of SEQ ID NO:20. In some embodiments, the nucleic acid molecule is or contains a nucleic acid sequence encoding the amino acid sequence set forth by of SEQ ID NO:21 or a nucleic acid sequence encoding an amino acid sequence having at least 90%, 91%, 92%, 93%, 94%, 95%, 96%, 97%, 98%, 99%, or 100% sequence identity to the sequence set forth by of SEQ ID NO:21. In some embodiments, the nucleic acid molecule is or contains a nucleic acid sequence encoding the amino acid sequence set forth by of SEQ ID NO:22 or a nucleic acid sequence encoding an amino acid sequence having at least 90%, 91%, 92%, 93%, 94%, 95%, 96%, 97%, 98%, 99%, or 100% sequence identity to the sequence set forth by of SEQ ID NO:22. In some embodiments, the nucleic acid molecule is or contains a nucleic acid sequence encoding the amino acid sequence set forth by of SEQ ID NO:23 or a nucleic acid sequence encoding an amino acid sequence having at least 90%, 91%, 92%, 93%, 94%, 95%, 96%, 97%, 98%, 99%, or 100% sequence identity to the sequence set forth by of SEQ ID NO: 23. It will be appreciated that due to the degeneracy of the genetic code, a plurality of nucleic acids may encode the same polypeptide.

In some embodiments, the nucleic acid hybridizes under stringent conditions to a nucleic acid encoding (or complementary to a nucleic acid encoding) an epimerase protein having at least 70%, 80%, 90%, 95%, 99%, or 100% sequence identity to the sequence set forth by SEQ ID NO: 2, SEQ ID NO:3, SEQ ID NO:4, SEQ ID NO:5, SEQ ID NO:6, SEQ ID NO:7, SEQ ID NO:8, or SEQ ID NO:9. In some embodiments, the nucleic acid hybridizes under stringent conditions to a nucleic acid encoding (or complementary to a nucleic acid encoding) an epimerase protein having at least 70%, 80%, 90%, 95%, 99%, or 100% sequence identity to the sequence set forth by SEQ ID NO:19, SEQ ID NO:20, SEQ ID NO:21, SEQ ID NO:22, or SEQ ID NO:23. In some embodiments, the nucleic acid hybridizes under stringent conditions to a nucleic acid encoding (or complementary to a nucleic acid encoding) an epimerase protein having at least 90%, 91%, 92%, 93%, 94%, 95%, 96%, 97%, 98%, 99%, or 100% sequence identity to the sequence set forth by SEQ ID NO:2, SEQ ID NO:3, SEQ ID NO:4, SEQ ID NO:5, SEQ ID NO:6, SEQ ID NO:7, SEQ ID NO:8, or SEQ ID NO:9. In some embodiments, the nucleic acid hybridizes under stringent conditions to a nucleic acid encoding (or complementary to a nucleic acid encoding) an epimerase protein having at least 90%, 91%, 92%, 93%, 94%, 95%, 96%, 97%, 98%, 99%, or 100% sequence identity to the sequence set forth by SEQ ID NO: 19, SEQ ID NO:20, SEQ ID NO:21, SEQ ID NO: 22, or SEQ ID NO:23.

In some embodiments, the nucleic acid molecule is or contains a nucleic acid sequence having at least 70%, 80%, 90%, 95%, 99%, or 100% sequence identity to the sequence set forth by SEQ ID NO:11, SEQ ID NO: 12, SEQ ID NO:13, SEQ ID NO: 14, SEQ ID NO: 15, SEQ ID NO: 16, SEQ ID NO:17, or SEQ ID NO:18. In some embodiments, the nucleic acid molecule is or contains a nucleic acid sequence having at least 70%, 80%, 90%, 95%, 99%, or 100% sequence identity to the sequence set forth by SEQ ID NO:24, SEQ ID NO:25, SEQ ID NO:26, SEQ ID NO: 27, or SEQ ID NO:28. In some embodiments, the nucleic acid molecule is or contains a nucleic acid sequence having at least 85%, 86%, 87%, 88%, 89%, 90%, 91%, 92%, 93%, 94%, 95%, 96%, 97%, 98%, 99%, or 100% sequence identity to the sequence set forth by SEQ ID NO: 11, SEQ ID NO:12, SEQ ID NO:13, SEQ ID NO:14, SEQ ID NO:15, SEQ ID NO:16, SEQ ID NO: 17, or SEQ ID NO:18. In some embodiments, the nucleic acid molecule is or contains a nucleic acid sequence having at least 85%, 86%, 87%, 88%, 89%, 90%, 91%, 92%, 93%, 94%, 95%, 96%, 97%, 98%, 99%, or 100% sequence identity to the sequence set forth by SEQ ID NO: 24, SEQ ID NO:25, SEQ ID NO:26, SEQ ID NO:27, or SEQ ID NO:28. In some embodiments, the nucleic acid molecule is or contains a nucleic acid sequence having at least 90%, 91%, 92%, 93%, 94%, 95%, 96%, 97%, 98%, 99%, or 100% sequence identity to the sequence set forth by SEQ ID NO:11, SEQ ID NO:12, SEQ ID NO:13, SEQ ID NO: 14, SEQ ID NO: 15, SEQ ID NO:16, SEQ ID NO:17, or SEQ ID NO:18. In some embodiments, the nucleic acid molecule is or contains a nucleic acid sequence having at least 90%, 91%, 92%, 93%, 94%, 95%, 96%, 97%, 98%, 99%, or 100% sequence identity to the sequence set forth by SEQ ID NO: 24, SEQ ID NO:25, SEQ ID NO:26, SEQ ID NO:27, or SEQ ID NO:28. In some embodiments, the nucleic acid molecule is or contains a nucleic acid sequence having the sequence set forth by SEQ ID NO: 11 or a nucleic acid sequence having at least 90%, 91%, 92%, 93%, 94%, 95%, 96%, 97%, 98%, 99%, or 100% sequence identity to the sequence set forth by SEQ ID NO: 11. In some embodiments, the nucleic acid molecule is or contains a nucleic acid sequence having the sequence set forth by SEQ ID NO: 12 or a nucleic acid sequence having at least 90%, 91%, 92%, 93%, 94%, 95%, 96%, 97%, 98%, 99%, or 100% sequence identity to the sequence set forth by SEQ ID NO:12. In some embodiments, the nucleic acid molecule is or contains a nucleic acid sequence having the sequence set forth by SEQ ID NO: 13 or a nucleic acid sequence having at least 90%, 91%, 92%, 93%, 94%, 95%, 96%, 97%, 98%, 99%, or 100% sequence identity to the sequence set forth by SEQ ID NO:13. In some embodiments, the nucleic acid molecule is or contains a nucleic acid sequence having the sequence set forth by SEQ ID NO:14 or a nucleic acid sequence having at least 90%, 91%, 92%, 93%, 94%, 95%, 96%, 97%, 98%, 99%, or 100% sequence identity to the sequence set forth by SEQ ID NO:14. In some embodiments, the nucleic acid molecule is or contains a nucleic acid sequence having the sequence set forth by SEQ ID NO: 15 or a nucleic acid sequence having at least 90%, 91%, 92%, 93%, 94%, 95%, 96%, 97%, 98%, 99%, or 100% sequence identity to the sequence set forth by SEQ ID NO: 15. In some embodiments, the nucleic acid molecule is or contains a nucleic acid sequence having the sequence set forth by SEQ ID NO: 16 or a nucleic acid sequence having at least 90%, 91%, 92%, 93%, 94%, 95%, 96%, 97%, 98%, 99%, or 100% sequence identity to the sequence set forth by SEQ ID NO:16. In some embodiments, the nucleic acid molecule is or contains a nucleic acid sequence having the sequence set forth by SEQ ID NO: 17 or a nucleic acid sequence having at least 90%, 91%, 92%, 93%, 94%, 95%, 96%, 97%, 98%, 99%, or 100% sequence identity to the sequence set forth by SEQ ID NO:17. In some embodiments, the nucleic acid molecule is or contains a nucleic acid sequence having the sequence set forth by SEQ ID NO:18 or a nucleic acid sequence having at least 90%, 91%, 92%, 93%, 94%, 95%, 96%, 97%, 98%, 99%, or 100% sequence identity to the sequence set forth by SEQ ID NO:18. In some embodiments, the nucleic acid molecule is or contains a nucleic acid sequence having the sequence set forth by SEQ ID NO:24 or a nucleic acid sequence having at least 90%, 91%, 92%, 93%, 94%, 95%, 96%, 97%, 98%, 99%, or 100% sequence identity to the sequence set forth by SEQ ID NO: 24. In some embodiments, the nucleic acid molecule is or contains a nucleic acid sequence having the sequence set forth by SEQ ID NO:25 or a nucleic acid sequence having at least 90%, 91%, 92%, 93%, 94%, 95%, 96%, 97%, 98%, 99%, or 100% sequence identity to the sequence set forth by SEQ ID NO:25. In some embodiments, the nucleic acid molecule is or contains a nucleic acid sequence having the sequence set forth by SEQ ID NO:26 or a nucleic acid sequence having at least 90%, 91%, 92%, 93%, 94%, 95%, 96%, 97%, 98%, 99%, or 100% sequence identity to the sequence set forth by SEQ ID NO:26. In some embodiments, the nucleic acid molecule is or contains a nucleic acid sequence having the sequence set forth by SEQ ID NO:27 or a nucleic acid sequence having at least 90%, 91%, 92%, 93%, 94%, 95%, 96%, 97%, 98%, 99%, or 100% sequence identity to the sequence set forth by SEQ ID NO:27. In some embodiments, the nucleic acid molecule is or contains a nucleic acid sequence having the sequence set forth by SEQ ID NO:28 or a nucleic acid sequence having at least 90%, 91%, 92%, 93%, 94%, 95%, 96%, 97%, 98%, 99%, or 100% sequence identity to the sequence set forth by SEQ ID NO: 28.

In some embodiments, the nucleic acid hybridizes under stringent conditions to the nucleic acid sequence set forth by SEQ ID NO: 11, SEQ ID NO:12, SEQ ID NO:13, SEQ ID NO:14, SEQ ID NO: 15, SEQ ID NO: 16, SEQ ID NO: 17, or SEQ ID NO: 18 or to a nucleic acid complementary to these nucleic acid sequences. In some embodiments, the nucleic acid hybridizes under stringent conditions to the nucleic acid sequence set forth by SEQ ID NO:24, SEQ ID NO:25, SEQ ID NO: 26, SEQ ID NO:27, or SEQ ID NO:28 or to a nucleic acid complementary to these nucleic acid sequences.

Nucleic acid molecules may encode a “full-length” (“fl” or “FL”) epimerase, which includes a signal sequence, only the mature form of an epimerase, which lacks the signal sequence, or a truncated form of an epimerase, which lacks the N or C-terminus of the mature form.

A nucleic acid molecule that encodes an epimerase can be operably linked to various promoters and regulators to drive expression when present, for example, in a host cell. Exemplary promoters are from B. licheniformis, B. subtilis, and Streptomyces. In some embodiments, the promoter is an aprE promoter. Such a nucleic acid molecule can also be linked to other coding sequences, e.g., to encode a chimeric polypeptide. In some embodiments, a nucleic acid sequence encoding an epimerase described herein operably linked to a heterologous promoter and/or regulator is referred to as a recombinant nucleic acid sequence.

In some embodiments, the nucleic acid molecules described herein may be present in a vector. The vector may be any vector into which the nucleic acid molecule can be inserted and which can be introduced into and optionally replicate within a host cell. In some embodiments, the vector may be referred to as an expression vector, meaning that the coding nucleic acid sequences contained in the vector are capable of in vivo or in vitro expression. The choice of vector, e.g. plasmid, cosmid, virus or phage vector, will often depend on the host cell into which it is to be introduced. In some embodiments, the vector is a plasmid.

In some cases, the vector may contain one or more selectable marker genes—such as a gene which confers antibiotic resistance e.g. ampicillin, kanamycin, chloramphenicol or tetracyclin resistance.

### C. Production of Epimerases

The epimerases described herein can be produced in host cells, for example, by secretion or intracellular expression, using methods well-known in the art. Suitable assays can be used to monitor epimerase activity in a sample, e.g., a media or cell sample (e.g., lysed cell sample) from a host cell culture. Fructose and/or allulose concentrations may be determined using high performance liquid chromatography (HPLC) or other means known in the art, e.g., a colorimetric assay. In some embodiments, production of epimerase occurs by liquid fermentation of host cells. See, e.g., Example 2.

Separation, isolation, and purification techniques are well known in the art and conventional methods can be used to extract epimerases from the host cell and/or media of culture conditions. In some embodiments, the host cells are used to produce a cultured cell material comprising an epimerase. In some embodiments, the cultured cell material is a cell lysate or a supernatant that includes the epimerase.

In aspects are provided host cells containing nucleic acid molecules or vectors as described in Section I-B. In some embodiments, the host cell is a yeast, a bacterium, a mammalian cell, or a plant cell. In some embodiments, the host cell is a yeast cell. In some embodiments, the host cell is a bacterium. In some embodiments, the host cell is a Bacillus spp. In some embodiments, the host cell is a B. subtilus or a B. lichenifomis. In some embodiments, the host cell is a B. subtilus.

### D. Epimerase Compositions

In some embodiments, the epimerase proteins provided herein are in soluble form. In some embodiments, the soluble protein may be used in reactors, such as columns, vessels, or tank reactors, to convert fructose, for example added as a part of a liquid substrate to the reactor, to allulose. In some embodiments, the soluble protein is contained in a composition including other proteins or enzymes, e.g., glucose isomerase. In some embodiments, the soluble protein is contained in a composition including other ingredients, e.g., metal ion cofactors.

In some embodiments, the epimerase protein described herein is immobilized on a matrix. Immobilization of the epimerase on a matrix is advantageous for increasing the usage life of the enzyme. In some cases, immobilization allows the protein to be used in industrial scale processes for commercial production of allulose. For example, matrix having protein immobilized thereon may be used in reactors, such as columns, vessels, or tank reactors, to convert fructose, for example added as a part of a liquid substrate to the reactor, to allulose.

In some embodiments, a produced epimerase, e.g., as described in Section I-C, is isolated and/or purified and immobilized on the matrix. In some embodiments, a host cell expressing the epimerase is immobilized on the matrix. For example, a host cell for production of the epimerase as described in Section I-C is immobilized on the matrix. In some embodiments, a broth containing lysed host cells used for producing the epimerase and expressed epimerase is immobilized on the matrix. In some embodiments, a cultured cell material comprising an epimerase is immobilized on the matrix. The matrix may be contacted with an isolated and/or purified epimerase, a host cell expressing an epimerase, a broth, and/or a cultured cell material such that at least the epimerase is immobilized on the matrix. In some embodiments, the matrix having the epimerase immobilized thereon is insoluble.

Exemplary matrixes include, but are not limited to, granules, beads, ion exchange resins, and polymer encapsulations. Non-limiting examples of matrixes contemplated herein as a suitable support include weak base polystyrene resins, weak base (—N(R)₂)phenol-formaldehyde resins, strong base (—N(R)₃)polystyrene resins, and/or miscellaneous enzyme adsorbants such as DEAE-Sephadex, DEAE-Glycophase, QAE-Glycophase, DEAE Bio-Gel A, CM Bio-Gel A, Selectacel DEAE-cellulose, Granular DEAE-cellulose, DEAE Sephacel, DEAE-Cellulose Beads, Controlled Pore Glass, Controlled Pore Aluminia, Titania, Zirconia (Corning Glass), bentonite, calcium carbonate.

In some embodiments, the epimerase is immobilized on a matrix as described in, for example, U.S. Pat. Nos. 3,796,634, 4,355,105, 4,713,333, 5,177,005, 5,437,993, 5,811,280, 5,916,789, and 7,297,510. In some embodiments, the epimerase is immobilized on a granule. In some embodiments, the granule is a colloidal particle. The granule may include a colloidal silica, activated charcoal, hydroxyapatite, alumina C gamma, bentonite, diatomaceous earth or a combination thereof. In some embodiments, the granule contains polyethylenimine (PEI). In some embodiments, the granule contains polyethylenimine (PEI) and glutaraldehyde. In some embodiments, the epimerase is immobilized on a bead. In some embodiments, the epimerase is immobilized on a resin. In some embodiments, the epimerase is immobilized on an ion exchange resin. In some embodiments, the epimerase is immobilized on a matrix by weakly basic ion exchange (i.e., electrostatic interaction based on the charge of the protein and the charge of a matrix such as a resin). Non-limiting examples of ion exchange resin include DuoLite™ and Amberlite™, e.g., as described herein. In some embodiments, the epimerase is immobilized by non-specific binding to porous regions of a matrix, such as a resin.

In an aspect is provided a conjugate including an epimerase and a matrix. A conjugate is a molecule comprised of two or more substructures bound together through a linking group to form a single structure. The binding can be made by connecting the subunits through a linking group. In some embodiments, the conjugate is formed by an amino group present in the epimerase reacting with an amine reactive material present on the matrix, e.g., glutaraldehyde. In some embodiments, the conjugate is a matrix having the epimerase immobilized thereon. In some embodiments, the conjugate is a granule, a glass bead, an ion exchange resin, or a polymer encapsulations having the epimerase immobilized thereon. In some embodiments, the conjugate is a granule having the epimerase immobilized thereon. In some embodiments, the conjugate is a resin, e.g., ion exchange resin, having the epimerase immobilized thereon. In some embodiments, the conjugate is insoluble.

### II. METHODS OF PRODUCING ALLULOSE

Also provided herein are methods of producing allulose using epimerases and compositions containing epimerases disclosed herein. In aspects, the method includes contacting an epimerase protein, e.g., a protein as described in Section I, with fructose or a substrate containing fructose. In some embodiments, the substrate containing fructose is a syrup, for example as described in Section II-A-2 below. In some embodiments, the contacting occurs under conditions where the fructose and allulose, e.g., as present in a substrate, are stable. For example, the contacting may occur at a temperature and/or pH that prevents or reduces the Maillard reaction and thus the browning of the sugars and/or favors allulose conversion. In some embodiments, the contacting occurs under conditions that are conducive to epimerase activity. For example, the contacting may occur at a temperature, a pH, and/or with a concentration of a metal cofactor that facilitates epimerase activity.

As described in Section I above, the epimerases provided herein may have a temperature range, pH range, and/or concentration of metal cofactor range in which activity, or an amount thereof, is retained. Likewise, fructose and allulose may have a temperature range and/or pH range that favors the conversion to allulose and the sugars are stable. Thus, in some cases, the contacting occurs under conditions, e.g., temperature, pH, metal cofactor content, where the equilibrium is favorable to the formation of allulose and the stability of the sugars and the activity of the epimerase overlap.

In some embodiments, the contacting occurs under conditions including a temperature in the range of about 50° C. to about 90° C. In some embodiments, the contacting occurs under conditions including a temperature in the range of about 50° C. to about 85° C. In some embodiments, the contacting occurs under conditions including a temperature in the range of about 50° C. to about 80° C. In some embodiments, the contacting occurs under conditions including a temperature in the range of about 50° C. to about 75° C. In some embodiments, the contacting occurs under conditions including a temperature in the range of about 50° C. to about 70° C. In some embodiments, the contacting occurs under conditions including a temperature in the range of about 60° C. to about 70° C. In some embodiments, the contacting occurs under conditions including a temperature of about 50° C. In some embodiments, the contacting occurs under conditions including a temperature of about 55° C. In some embodiments, the contacting occurs under conditions including a temperature of about 60° C. In some embodiments, the contacting occurs under conditions including a temperature of about 65° C. In some embodiments, the contacting occurs under conditions including a temperature of about 70° C. In some embodiments, the contacting occurs under conditions including a temperature of about 75° C. It is also possible for the contacting to occur at an initial temperature, e.g., 50° C., when the enzymes are first contacted with fructose or the substrate containing fructose, and ramped to higher temperatures, e.g., up to 70° C., for subsequent contacting with fructose or the substrate containing fructose. For example, as the enzyme is used over weeks and/or months, either continuously or intermittently as required by the production process, the temperature conditions for contacting may be increased incrementally over time.

In some embodiments, the contacting occurs under conditions including a pH in a range of about 4 to about 10. In some embodiments, the contacting occurs under conditions including a pH in a range of about 4 to about 9. In some embodiments, the contacting occurs under conditions including a pH in a range of about 4 to about 8. In some embodiments, the contacting occurs under conditions including a pH in a range of about 4.5 to about 8. In some embodiments, the contacting occurs under conditions including a pH in a range of about 5 to about 8. In some embodiments, the contacting occurs under conditions including a pH in a range of about 5 to about 7.5. In some embodiments, the contacting occurs under conditions including a pH in a range of about 5 to about 7. In some embodiments, the contacting occurs under conditions including a pH in a range of about 5 to about 6.5. In some embodiments, the contacting occurs under conditions including a pH in a range of about 5 to about 6. In some embodiments, the contacting occurs under conditions including a pH of about 5. In some embodiments, the contacting occurs under conditions including a pH of about 5.5. In some embodiments, the contacting occurs under conditions including a pH of about 6. In some embodiments, the contacting occurs under conditions including a pH of about 6.5. In some embodiments, the contacting occurs under conditions including a pH of about 7. In some embodiments, the contacting occurs under conditions including a pH of about 7.5. It is also possible for the contacting to occur at an initial pH, e.g., 5, when the enzymes are first contacted with fructose or the substrate containing fructose, and ramped to more basic pHs, e.g., up to 10, for subsequent contacts with fructose or the substrate containing fructose. For example, as the enzyme is used over weeks and/or months, either continuously or intermittently as required by the production process, the pH conditions for contacting may be increased (become more basic) incrementally over time.

In some embodiments, the contacting occurs under conditions where a metal cofactor is added (supplemented) to reach a concentration in a range of about 0 to about 2 mM. In some embodiments, the contacting occurs under conditions where a metal cofactor is added (supplemented) to reach a concentration in a range of about 0 to about 1 mM. In some embodiments, the contacting occurs under conditions where a metal cofactor is added (supplemented) to reach a concentration in a range of about 0 to about 0.5 mM. In some embodiments, the contacting occurs under conditions where a metal cofactor is added (supplemented) to reach a concentration in a range of about 0 to about 0.25 mM. In some embodiments, the contacting occurs under conditions where a metal cofactor is added (supplemented) to reach a concentration in a range of about 0 to about 0.15 mM. In some embodiments, the contacting occurs under conditions where a metal cofactor is added (supplemented) to reach a concentration in a range of about 1 mM to about 2 mM. In some embodiments, the metal cofactor is in the concentration in a range of about 1 to about 2 mM when the metal cofactor is in the form of a salt. In some embodiments, the contacting occurs under conditions including a metal cofactor at a concentration in a range of about 0.15 to about 0.25 mM. In some embodiments, the metal cofactor is in the concentration in a range of about 0.15 to about 0.25 mM when the metal cofactor is not in salt form, e.g., present as an ion. In any of the cases where metal cofactor is added, the amount of metal cofactor that is added is less than the target concentration. In some embodiments, the contacting occurs under conditions where a metal cofactor is not added (supplemented). In some embodiments, the concentration of the metal cofactor exists without the need to supplement the metal cofactor. In some embodiments, the cofactor is an ion. In some embodiments, the cofactor is a salt. In some embodiments, the metal cofactor is magnesium or a salt thereof.

### A. Fructose, Substrates Containing Fructose, and Conversion to Allulose

In some embodiments, the fructose or substrate containing fructose is produced as part of a carbohydrate production process. In some embodiments, the methods for producing allulose provided herein are implemented as part of a carbohydrate production process. In some embodiments, the carbohydrate production process is a production process implemented at a biorefinery.

Those of general skill in the art are well aware of available methods that may be used to prepare fructose and substrates containing fructose for use in the methods disclosed herein. Methods of preparation generally include process steps such as milling/grinding, liquefaction, saccharification, and isomerization for converting biomass to a sugar (e.g., a syrup).

### 1. Milling

Fructose and substrates containing fructose may be obtained from tubers, roots, stems, legumes, cereals or whole grain by processing derived starches. In some embodiments, the starch, and subsequently the fructose or substrate containing fructose, may be obtained from corn, cobs, sugar cane, sugar beets, wheat, barley, rye, triticale, milo, sago, millet, cassava, tapioca, sorghum, rice, peas, bean, banana, or potatoes. In some embodiments, the fructose or substrate containing fructose is obtained from processing starch from corn or cobs.

Starch from a grain may be ground or whole and may include solids, such as corn kernels, bran and/or cobs. The starch may also be highly refined raw starch or feedstock from starch refinery processes. Various starches and fructose also are commercially available.

The starch may be a crude starch from milled whole grain, which contains non-starch fractions, e.g., germ residues and fibers. Milling may comprise either wet milling or dry milling or grinding. In wet milling, whole grain is soaked in water or dilute acid to separate the grain into its component parts, e.g., starch, protein, germ, oil, kernel fibers. Wet milling efficiently separates the germ and meal (i.e., starch granules and protein) and is especially suitable for production of syrups.

In dry milling or grinding, whole kernels are ground into a fine powder and often processed without fractionating the grain into its component parts. In some cases, oils and/or fiber from the kernels are recovered. Dry ground grain thus will comprise significant amounts of non-starch carbohydrate compounds, in addition to starch. Dry grinding of the starch substrate can be used for production of ethanol and other biochemicals.

### 2. Liquefaction

Liquefaction refers to a process by which starch is converted to less viscous and shorter chain dextrins. Generally, this process involves gelatinization of starch simultaneously with or followed by the addition of an α-amylase, although additional liquefaction-inducing enzymes optionally may be added. The starch substrate is generally slurried with water. The starch slurry may contain starch as a weight percent of dry solids of about 10-55%, about 20-45%, about 30-45%, about 30-40%, or about 30-35%. The α-amylase typically used for this application is thermally stable. The α-amylase is usually supplied, for example, at about 1500 units per kg dry matter of starch. To optimize α-amylase stability and activity, the pH of the slurry typically is adjusted to about pH 4.5-6.5 and about 1 mM of calcium (about 40 ppm free calcium ions) can also be added, depending upon the properties of the amylase used. Bacterial α-amylase remaining in the slurry following liquefaction may be deactivated via a number of methods, including lowering the pH in a subsequent reaction step or by removing calcium from the slurry in cases where the enzyme is dependent upon calcium.

The slurry of starch plus α-amylase may be pumped continuously through a jet cooker, which is steam heated to a temperature in the range of about 105° C. to 110° C. Gelatinization occurs rapidly under these conditions, and the enzymatic activity, combined with the significant shear forces, begins the hydrolysis of the starch substrate. The residence time in the jet cooker is brief, e.g., anywhere in the range of about 4 to about 12 minutes. The partly gelatinized starch may be passed into a series of holding tubes maintained at 105-110° C. and held for 5-8 min. to complete the gelatinization process (“primary liquefaction”). Hydrolysis to the required DE is completed in holding tanks at 85-95° C. or higher temperatures for about 1 to 2 hours (“secondary liquefaction”). The slurry is then allowed to cool to room temperature. This cooling step can be 30 minutes to 180 minutes, e.g., 90 minutes to 120 minutes. The liquefied starch typically is in the form of a slurry having a dry solids content (w/w) of about 10-50%; about 10-45%; about 15-40%; about 20-40%; about 25-40%; or about 25-35%.

### 3. Saccharification and Isomerization

Liquefied starch can be saccharified into a syrup rich in lower DP (e.g., DP1+DP2) saccharides, using glucoamylases, optionally in the presence of another enzyme(s). Exemplary DP1 saccharides include glucose and fructose, and DP2 saccharides include, for example, maltose and sucrose. Depending on the enzymes used, syrups may contain a weight percent of DP2 of the total oligosaccharides in the saccharified starch exceeding 30%, e.g., 45%-65% or 55%-65%. The weight percent of (DP1+DP2) in the saccharified starch may exceed about 70%, e.g., 75%-85% or 80%-85%.

In some embodiments, an isomerization step may be used to modify the composition of lower DP in the syrup. In some embodiments, enzymes may be used to increase the amount of fructose or DP1 saccharides capable of being converted to fructose, e.g., glucose, in the syrup. However, any method of increasing the DP1 content of syrup is contemplated as useful for the methods of converting D-fructose to allulose provided herein since DP1 saccharides, such as glucose and fructose, can be converted either indirectly or directly to allulose. For example, a syrup may be contacted with the epimerases described herein allowing for the direct conversion of fructose present in the syrup to allulose. In some cases, the syrup may be contacted with a glucose isomerase enzyme to convert glucose present in the syrup to fructose, which can in turn be converted to allulose by contact with the provided epimerases.

In some embodiments, the substrate containing fructose is a syrup. In some embodiments, the syrup is high fructose corn syrup (HFCS). In some embodiments, the substrate containing fructose is a syrup that does not contain fructose, but contains saccharides, e.g., DP1 saccharides, capable of being converted to fructose. In some embodiments, the substrate containing fructose is a syrup including DP1 saccharides that may be converted to fructose. In some embodiments, the substrate containing fructose is a syrup containing fructose and DP1 saccharides that may be converted to fructose. In some embodiments, the DP1 saccharides is glucose. In some embodiments, the conversion of glucose to fructose is accomplished by enzymes, e.g., glucose isomerases.

The method of producing allulose from fructose or a substrate containing fructose, e.g., a syrup, may proceed by contacting an epimerase protein described herein with the fructose or the substrate containing fructose. In this way, the fructose or fructose contained in the substrate is converted to allulose. In some embodiments, when the substrate containing fructose includes only or further includes glucose, the substrate may be contacted or also contacted with a glucose isomerase to convert the glucose to fructose. Suitable isomerases for conversion of glucose to fructose include, but are not limited to, SWEETZYME® IT, IT Extra, T (Novozymes A/S); G-ZYME® IMGI, and G-ZYME® G993, KETOMAX®, G-ZYMER G993, G-ZYMER G993 liquid; GENSWEET® IGI (SA, HF, VHF, MAX); and GENSWEET® SGI. Following isomerization of glucose, the mixture typically contains about 40-45% fructose, e.g., 42% fructose. In some cases, the mixture may be further isolated or purified to increase the percentage of fructose. In some embodiments, the mixture may be purified to contain about or at least 95% fructose. The substrate containing fructose obtained from the converted glucose, may be contacted with an epimerase described herein to convert the fructose to allulose. In some embodiments, the substrate is contacted with the glucose isomerase and subsequently contacted with the epimerase. In some embodiments, the substrate is contacted with the glucose isomerase and the epimerase simultaneously. It should be appreciated that in some cases a metal cofactor may be added to facilitate the activity of the glucose isomerase.

In some embodiments, the epimerase with which the fructose or substrate containing fructose is contacted is in soluble form. In some embodiments, the epimerase with which the fructose or substrate containing fructose is contacted is immobilized on a matrix. See, e.g., Section I-D. In some embodiments, for example when the substrate containing fructose is contacted with a glucose isomerase, the glucose isomerase is immobilized on a matrix. In some embodiments, the matrix is a granule. In some embodiments, the matrix is an ion exchange resin.

In some embodiments, the epimerase is immobilized on a first matrix and the glucose isomerase is immobilized on a second matrix. In some embodiments, the first matrix and the second matrix are made of different material. In some embodiments, the first matrix and the second matrix are made of the same material. In some embodiments, the first matrix and second matrix are granules. In some embodiments, the first matrix and second matrix are ion exchange resins. In some embodiments, the epimerase and the glucose isomerase are co-immobilized on a matrix. In some embodiments, the matrix is a granule. In some embodiments, the matrix is an ion exchange resin.

Various advantages are associated with the use of immobilized proteins, including, but not limited to, a longer duration of use of the proteins and avoiding a step of inactivating or removing proteins from the product, for example when the immobilized proteins are contained in a reactor that allows substrate to contact the immobilized protein and be collected. Thus, in some embodiments, the protein, e.g., epimerase and/or glucose isomerase, is immobilized on a matrix which is present, e.g., loaded or packed, in a reactor. In some embodiments, the protein, e.g., epimerase and/or glucose isomerase, is in a soluble form and present in a reactor. In some embodiments, contacting the protein with the substrate containing fructose occurs by adding the substrate to the reactor. In some embodiments, the reactor is a column, a tank, or a vessel. In some embodiments, the reactor is a column. Non-limiting examples of columns contemplated for use herein include fixed-bed columns and fluidized bed columns. In some embodiments, the substrate is allowed to pass through the column and is collected. In some embodiments, the reactor is a tank or vessel. Non-limiting examples of tanks and vessels contemplated for use herein include fluidized bed tanks, stirred tanks, and stirred vessels. In some embodiments, the substrate is collected from the tank or reactor following contact with the immobilized protein.

In some embodiments, the reactor contains a matrix on which the epimerase is immobilized. In some embodiments, the reactor contains a matrix on which the glucose isomerase is immobilized. In some embodiments, the reactor contains a first matrix on which the epimerase is immobilized and a second matrix on which the glucose isomerase is immobilized. In some embodiments, the reactor contains a matrix on which the epimerase and glucose isomerase are co-immobilized. It should be understood that the number and configuration of reactors depends on the composition of the substrate and whether sequential or simultaneous contacting of the substrate with the proteins is preferred.

In some embodiments, the substrate contacted with the protein is collected from the reactor. For example, if using a column, the substrate may be added at one end of the column, allowed to pass through the matrix having the immobilized protein, and collected at the other end. In some embodiments, the collected substrate contains fructose which may be optionally purified and passed to another reactor containing matrix with immobilized epimerase to facilitate the conversion to allulose. In some embodiments, the collected substrate contains allulose. In some embodiments, the allulose is purified from the collected substrate.

### III. KITS

Also provided are kits including the compositions, e.g., epimerase proteins, immobilized epimerase proteins, nucleic acid sequences, vectors, host cells, and/or cultured cell material comprising epimerase described herein, which may further include instructions on methods of using the compositions, such as uses described herein. In some embodiments, the kit includes epimerase proteins as described herein. In some embodiments, the kit includes polynucleotides encoding epimerases as described herein. In some embodiments, the kit includes vectors encoding epimerases as described herein. In some embodiments, the kit includes cell culture material comprising epimerase as described herein. In some embodiments, the kit includes an epimerase immobilized on a matrix as described herein. In some embodiments, the kit includes a conjugate as described herein. The kits may also include other materials desirable from a commercial and user standpoint, including other buffers, diluents, filters, needles, syringes, columns (e.g., reactor columns), vessels (e.g., reactor vessels), and package inserts with instructions for use.

### IV. EXEMPLARY EMBODIMENTS

Among the provided embodiments are:
1. A protein comprising an amino acid sequence having at least 70% sequence identity to the sequence set forth by SEQ ID NO:2, SEQ ID NO:3, SEQ ID NO:4, SEQ ID NO:5, SEQ ID NO:6, SEQ ID NO:7, SEQ ID NO:8, or SEQ ID NO:9, wherein the protein has epimerase activity.
2. The protein of embodiment 1, wherein the amino acid sequence has at least 80%, 90%, 95%, 99%, or 100% sequence identity to the sequence set forth by SEQ ID NO:2, SEQ ID NO:3, SEQ ID NO: 4, SEQ ID NO:5, SEQ ID NO:6, SEQ ID NO:7, SEQ ID NO:8, or SEQ ID NO:9.
3. The protein of embodiment 1 or embodiment 2, wherein the protein is immobilized on a matrix.
4. The protein of embodiment 3, wherein the matrix is a granule or an ion exchange resin.
5. A nucleic acid molecule comprising a nucleic acid sequence encoding a protein according to any one of embodiments 1-4.
6. The nucleic acid molecule of embodiment 5, comprising a nucleic acid sequence that: i) encodes an amino acid sequence having at least 90%, 95%, 99%, or 100% sequence identity to the sequence set forth by SEQ ID NO:2, SEQ ID NO:3, SEQ ID NO:4, SEQ ID NO:5, SEQ ID NO:6, SEQ ID NO:7, SEQ ID NO:8, or SEQ ID NO:9; ii) has at least 70%, 80%, 90%, 95%, 99%, or 100% sequence identity to the sequence set forth by SEQ ID NO:11, SEQ ID NO:12, SEQ ID NO: 13, SEQ ID NO:14, SEQ ID NO:15, SEQ ID NO:16, SEQ ID NO:17, or SEQ ID NO:18; or iii) hybridizes under stringent conditions to a nucleic acid sequence having a sequence complementary to the sequence set forth by SEQ ID NO:11, SEQ ID NO:12, SEQ ID NO:13, SEQ ID NO:14, SEQ ID NO:15, SEQ ID NO:16, SEQ ID NO:17, or SEQ ID NO:18.
7. The nucleic acid molecule of embodiment 5 or embodiment 6, comprising a heterologous regulatory sequence, optionally a promoter sequence.
8. A vector comprising a nucleic acid molecule according to any one of embodiments 5-7.
9. A host cell comprising a nucleic acid molecule according to any one of embodiments 5-7 or a vector according to embodiment 8.
10. The host cell of embodiment 9, wherein the host cell is a yeast, a bacterium, a mammalian cell, or a plant cell.
11. The host cell of embodiment 9 or embodiment 10, wherein the host cell is Bacillus subtilis.
12. A cultured cell material comprising a protein of embodiment 1 or embodiment 2 and/or a host cell of any one of embodiments 9-11.
13. Allulose produced by a protein according to any one of embodiments 1-4.
14. A composition for producing allulose comprising: i) a protein according to any one of embodiments 1-4; and ii) a substrate comprising fructose.
15. The composition of embodiment 14, wherein the protein is immobilized on a matrix.
16. The composition of embodiment 14 or embodiment 15, wherein the substrate comprises glucose.
17. The composition of any one of embodiments 14-16, further comprising a glucose isomerase immobilized on a matrix.
18. The composition of any one of embodiments 14-17, wherein the protein and glucose isomerase are co-immobilized on the same matrix or immobilized on different matrixes.
19. The composition of any one of embodiments 14-18, wherein the composition is comprised in a reactor.
20. Use of a protein according to any one of embodiments 1˜4 for producing allulose.
21. A method of producing allulose, comprising contacting a protein according to any one of embodiments 1˜4 with a substrate comprising fructose.
22. The method of embodiment 21, wherein the contacting occurs under conditions comprising a temperature in a range of about 50° C. to about 90° C.
23. The method of embodiment 21 or embodiment 22, wherein the contacting occurs under conditions comprising a pH in a range of about 4.5 to about 8.
24. The method of any one of embodiments 21-23, wherein the contacting occurs under conditions where no metal cofactor is added or an amount of metal cofactor that is less than a metal cofactor concentration needed for epimerase activity is added.
25. The method of any one of embodiments 21-24, wherein the protein is immobilized on a matrix comprised in a reactor, and the contacting occurs by adding the substrate comprising fructose to the reactor.
26. The method of any one of embodiments 21-25, wherein the substrate comprising fructose is produced by: (i) contacting a substrate comprising glucose with a glucose isomerase prior to contacting the protein; or (ii) contacting a substrate comprising glucose with a glucose isomerase at the same time as contacting the protein.
27. The method of any one of embodiments 21-26, comprising purifying the produced allulose.
28. A kit comprising: (i) a protein according to any one of embodiments 1-4, a nucleic acid molecule according to any one of embodiments 5-7, a vector according to embodiment 8, a host cell according to any one of embodiments 9-11, and/or a cell culture material according to embodiment 12; and (ii) instructions for use.

### V. EXAMPLES

The following examples are included for illustrative purposes only and are not intended to limit the scope of the invention.

### Example 1: Expression of D-allulose 3-epimerases

Amino acid sequences of D-allulose 3-epimerases were found in the NCBI database (Accession numbers are shown in Table E1). The expression vector p2JM103BBI (Vogtentanz, Protein Expr Purif, 55:40-52, 2007) was employed for the expression of all listed exemplary epimerases in Bacillus subtilis. The plasmid contained an aprE promoter followed by a codon-optimized nucleotide sequence encoding the protein sequence of the target gene. The corresponding protein sequences (SEQ ID NOs: 1-9, 19-23) and codon-optimized gene sequences (SEQ ID NOs: 10-18, 24-28) are shown in Table E1.

Competent B. subtilis cells were transformed and plated on Luria Agar plates supplemented with 5 ppm chloramphenicol. Colonies were picked and subjected to fermentation in a 250 ml shake flask with MBD medium (a MOPS based defined medium, supplemented with additional 5 mM CaCl₂)). Supernatants from these cultures were used to confirm the protein expression by SDS-PAGE analysis and assay for enzyme activity.

TABLE E1
Exemplary epimerases

| SEQ ID | | |
|---|---|---|
| NO | Sequence | Description |
| 1 | MKIGCHGLVWTGHFDAEGIRYSVQKTREAGFDLVEFPLMDPFSFDVQTAKS | AglEpi, NCBI |
| | ALAEHGLAASASLGLSDATDVSSEDPAVVKAGEELLNRAVDVLAELGATDFC | Accession No. |
| | GVIYSAMKKYMEPATAAGLANSKAAVGRVADRASDLGINVSLEVVNRYETN | BAW27657.1 |
| | VLNTGRQALAYLEELNRPNLGIHLDTYHMNIEESDMFSPILDTAEALRYVHIG | |
| | ESHRGYLGTGSVDFDTFFKALGRIGYDGPVVFESFSSSVVAPDLSRMLGIWR | |
| | NLWADNEELGAHANAFIRDKLTAIKTIELH | |
| 2 | MKYGIYYSYWEESWGADFVKYIEKVSKLGFDVLEINASALFDYSDNKINEVKK | BsuEpi1, NCBI |
| | IINDNNIILTAGYGPIAEHNIGNINTRNKALDWYKKVFSVMQKLDIHIICGAIY | Accession No. |
| | SYWPVDYSKKINKEEDLKMSIEGMKLLANIAKEYNVNICCEVLNRFENYLLNT | WP_048595324.1 |
| | AKEAVAFVKEVGFDNVKVMLDTFHMNIEEESFANAIKEAGKYLGHFHLGEP | |
| | NRRVPGEGRMPWNEILDALISIDYKGYAVMEPFVKRGGEVETDIKIWRDLV | |
| | ADTSEEKLDNAAKNSVHFLKTLEKIRRELL | |
| 3 | MKYGIYYAYWEKEWRGDFIPYIEKVKKLGFDVLEVACGDFHNQPDSYFHAL | CbaEpi17, NCBI |
| | RDAAKEYDITLTGGYGPRAEHNLASKDPAVVENALAFYSKIFSKMDMAGIRS | Accession No. |
| | IGGALYSYWPVDYSRDPDKEGDLERSIRGMQRLADIAARYGITLNMEVLNRF | NLW02352.1 |
| | EGYLINDAREGLSYIRAVDKSNVKLMLDTFHMNIEEDSLTEPILLAGKDLGHL | |
| | HVGEPNRKPPREGRMPWKEIGQALRQIGYEGAVVMEPFVTMGGQVGKDI | |
| | RVWRDISGGATRSDLDRDAAKSVSFLRSMFEG | |
| 4 | MKYGIYYAYWEKEWGGDFIPYVAKVKQLGFDVLEVACGDFHNQPDSYLHA | CbaEpi18, NCBI |
| | LRETAKENDIILTGGYGPRAEHNLASPDPAVVENALAFYSEIFRKMDIADIRSI | Accession No. |
| | GGALYSYWPIDYNKELDKDGDLERSIQGMRKLADIAGRYGITLNMEVLNRFE | NLI38423.1 |
| | GYLINDVREGLAYVRAVDKPNVKLMLDTFHMNIEEDSLIEPILLAGKDLGHV | |
| | HVGEPNRKPPREGRMPWKEIGVALREIGYDGAVVMEPFVTMGGQVGKDI | |
| | RIWRDISQGATENDLDRDAAKSVAFLRSAFEE | |
| 5 | MPSLIGCHALVWTGEFDAAGITSSVERTKAAGFDLVEFPLMDPYTFDAATA | NdeEpi1, NCBI |
| | RKALGDADLTGSASLGLPEHADISSEDPAVVAAGVDLLRTALDRVAEMGGT | Accession No. |
| | RLCGVLYGSMRKHMAPLSERGRANSVAAIRDLAGRAAAVGVHLSLEVVNRY | WP_111768618.1 |
| | ETNVFNTGRGALAYLDAVADTNVSVHLDTYHMNIEESDLFQPFLDTSERLGY | |
| | VHIGESHRGYLGTGSVDFESTFRALARTGYDGPIVFESFSSAVVSPTLSNTLGI | |
| | WRNLWTDSEDLAAHANRFIRDHIAAVRTIDLH | |
| 6 | MQIGVHALVFTGTFDRDGISLAVEKTAAAGFDLIEFPLMDPFAFDREHAAKE | LphEpi1, NCBI |
| | LAAHGIAATASLGLSDATDLTSDDPAVRAAGERLLEECLDIVHALGATKLCGV | Accession No. |
| | LYSAMKKYVTPPTAEGRAASAAAIGRLAEKAADLGIDLSLEVVNRYETNLINT | WP_128493859.1 |
| | GRQALGFLDEIGRGNVFVHLDTYHMNIEESDLIQPVLDVDRRLGYVHIGESH | |
| | RGYLGSGTVDFDSFFRGLARVGYDGPVVFESFSSTVVSATLSNTLGVWRNL | |
| | WDDGDDLAAHANRFIRDGLRTVETIALH | |
| 7 | MLIGAHALVFSGSFDAAGLTLAIEKTKQAGFDLIEIPLMDPFHFDRMLAARLL | MspEpi3, NCBI |
| | QENELAVSASLGLSEATDLTSNDPDVVARGERMLEECLEIVHAMGGSDLCG | Accession No. |
| | VIYSAMKKYMAPATPAGRASSAAALARLASRAEGLGIRLSLEVVNRYETNLM | WP_108392186.1 |
| | NTGRQALAFLDEVGHDNVSVHLDTYHMNIEESDLYTPVLDVGNRLGYAHV | |
| | GESHRGYLGSGTVDFGSFFRGLARIGYDGPVVFESFSSAVVNEELSRMLGIW | |
| | RNLWDDPDDLGAHANRFIRTQLRAVETIALH | |
| 8 | MKYGIYYAYWEKEWTGDYFKYIDKVKELGFDILEISCGAFKKDYTTDEQLLRI | OsiEpi1, NCBI |
| | GEYAKEKGIILTAGYGPQKDENLASPDEAVQKNAIEFFKETLRKLHLMNIHILG | Accession No. |
| | GGLYSYWPVDFNSDINKERDFETSVKNMRLIAKYAEEYDITLGMEVLNRFEG | WP_183684385.1 |
| | YLINTCDECLRYIDAVGNNRVGVMLDTFHMNIEEDNIGDAIRKAGDKLVHF | |
| | HMGERNRKVPGKGTMPWAEIGQALRDIHYQHAAVMEPFVLOGGKVGQD | |
| | IKIWRNLLPDTSEAKLDADAKHSCEFVRHVFEL | |
| 9 | MLIGAHALVFTGSFEQAGLTTAIEKTKQAGFDLIEIPLMDPANFDRMLAARLL | MspEpi4, NCBI |
| | RDNDLAVSASLGLSEATDLTSGDPAVVAAGERMLEECIDIVQAMGGTRLCG | Accession No. |
| | VIYSAMKKYMEPVTASGRASSAAALGRLASRAEDAGVHLSLEVVNRYETNV | WP_196832999.1 |
| | MNTGRQALGFLDEIGHSNVSVHLDTYHMNIEESDLYTPVLDVDTRLGYVHI | |
| | GESHRGYLGSGTVDFGSFFRGLARVSYDGPVVFESFSSAVVNQDLSRMLGI | |
| | WRNLWDDSDDLAAHANRFIRDQLRAVESISLH | |
| 10 | ATGAAAATTGGCTGCCATGGCTTAGTGTGGACAGGACATTTTGATGCGG | AglEpi |
| | AAGGCATCCGTTATTCAGTCCAAAAAACACGCGAAGCAGGCTTTGATCTT | |
| | GTGGAATTTCCTCTTATGGATCCGTTTAGCTTTGATGTTCAAACAGCGAA | |
| | ATCAGCACTTGCAGAACATGGCCTTGCAGCGTCTGCGAGCCTGGGACTG | |
| | TCAGATGCAACGGATGTCTCTTCTGAAGATCCGGCGGTGGTCAAAGCGG | |
| | GAGAAGAATTACTTAATCGTGCAGTCGATGTGCTTGCGGAATTAGGAGC | |
| | AACGGATTTTTGCGGAGTTATCTATAGCGCAATGAAAAAATATATGGAAC | |
| | CTGCGACAGCAGCAGGCCTTGCAAATTCTAAAGCGGCGGTCGGCCGGGT | |
| | CGCGGATCGCGCGTCAGATTTAGGCATCAATGTCTCACTGGAAGTGGTC | |
| | AATCGGTATGAAACGAATGTCCTTAATACAGGACGCCAAGCGCTGGCGT | |
| | ATCTTGAAGAACTTAATAGACCTAATCTGGGCATTCATTTAGATACGTATC | |
| | ATATGAATATCGAAGAAAGCGATATGTTTAGCCCTATCCTGGATACGGCA | |
| | GAAGCACTTCGGTATGTCCATATTGGAGAATCACATAGAGGCTATTTAGG | |
| | AACGGGCTCAGTCGATTTTGATACGTTTTTTAAAGCGTTGGGACGCATTG | |
| | GCTATGATGGCCCGGTTGTGTTTGAGAGCTTTTCTAGCTCAGTTGTCGCA | |
| | CCTGATCTTTCTCGCATGCTGGGCATCTGGCGTAATCTGTGGGCAGATAA | |
| | TGAAGAACTGGGAGCACATGCAAATGCGTTTATTCGTGATAAACTGACG | |
| | GCGATTAAAACAATCGAACTTCAT | |
| 11 | ATGAAATATGGCATCTATTATAGCTATTGGGAAGAATCTTGGGGCGCAG | Bsu Epi1 |
| | ATTTTGTTAAATATATCGAAAAAGTTTCTAAACTTGGCTTTGATGTTCTGG | |
| | AAATCAATGCAAGCGCACTGTTTGATTATAGCGATAATAAAATCAATGAA | |
| | GTTAAAAAAATCATCAATGATAATAATATCATCCTGACGGCAGGCTATGG | |
| | CCCGATCGCAGAACATAATATCGGCAATATCAATACACGCAATAAAGCAT | |
| | TAGATTGGTATAAAAAAGTTTTTAGCGTTATGCAAAAACTTGACATACAT | |
| | ATCATCTGCGGAGCAATTTATAGCTATTGGCCGGTTGATTATTCTAAAAA | |
| | AATTAATAAAGAAGAAGATCTTAAAATGTCTATTGAAGGCATGAAACTGC | |
| | TGGCAAATATCGCAAAAGAATATAATGTTAATATCTGCTGCGAAGTTCTG | |
| | AATCGCTTTGAAAATTATTTACTGAATACAGCAAAAGAAGCAGTTGCATT | |
| | TGTTAAAGAAGTTGGCTTTGATAATGTTAAAGTTATGCTGGATACGTTTC | |
| | ATATGAATATCGAAGAAGAGTCGTTTGCAAATGCAATCAAAGAAGCAGG | |
| | CAAATATTTAGGACATTTTCATTTAGGCGAACCTAATAGACGCGTTCCGG | |
| | GCGAAGGACGCATGCCGTGGAATGAAATCCTGGATGCACTGATCAGCAT | |
| | CGATTATAAAGGCTATGCAGTTATGGAACCGTTTGTTAAACGCGGAGGA | |
| | GAAGTTGAAACGGACATAAAAATCTGGAGAGATCTGGTTGCAGATACGA | |
| | GCGAAGAAAAACTTGATAATGCAGCAAAAAATTCAGTTCATTTTCTTAAA | |
| | ACACTGGAAAAAATTCGCCGCGAACTGCTG | |
| 12 | ATGAAATATGGCATCTATTATGCATATTGGGAAAAAGAATGGAGAGGCG | CbaEpi17 |
| | ATTTTATCCCGTATATCGAAAAAGTTAAAAAACTGGGCTTTGATGTTCTG | |
| | GAAGTTGCATGCGGAGATTTTCATAATCAACCGGATTCTTATTTTCATGCA | |
| | CTGCGCGATGCAGCAAAAGAATATGACATAACACTGACGGGAGGCTATG | |
| | GCCCTCGCGCAGAACATAATTTAGCATCTAAAGATCCGGCAGTTGTTGAA | |
| | AATGCACTTGCATTTTATTCTAAAATCTTTTCTAAAATGGATATGGCAGGC | |
| | ATCCGCTCAATCGGCGGCGCACTGTATAGCTATTGGCCGGTTGATTATAG | |
| | CCGCGATCCGGATAAAGAAGGAGATTTAGAACGCTCAATTAGAGGAATG | |
| | CAACGCCTTGCAGACATCGCAGCACGCTATGGAATTACACTTAATATGGA | |
| | AGTTCTTAATCGCTTTGAAGGCTATCTGATCAATGATGCACGCGAAGGAC | |
| | TGTCTTATATCAGAGCAGTTGATAAATCTAATGTTAAACTGATGCTGGAT | |
| | ACGTTTCATATGAATATCGAAGAAGATAGCCTGACGGAACCGATCCTGCT | |
| | GGCAGGCAAAGATCTGGGACATTTACATGTTGGCGAACCTAATCGCAAA | |
| | CCTCCTAGAGAAGGCCGCATGCCGTGGAAAGAAATTGGACAAGCACTTA | |
| | GACAAATCGGCTATGAAGGAGCAGTTGTTATGGAACCGTTTGTTACGAT | |
| | GGGCGGACAAGTTGGCAAAGATATTCGCGTTTGGCGCGACATAAGCGG | |
| | CGGAGCAACACGCTCAGATCTTGATAGAGATGCAGCAAAATCAGTTAGC | |
| | TTTCTGCGCTCAATGTTTGAAGGA | |
| 13 | ATGAAATATGGCATCTATTATGCATATTGGGAAAAAGAATGGGGCGGAG | CbaEpi18 |
| | ATTTTATTCCGTATGTTGCAAAAGTTAAACAATTAGGCTTTGATGTTCTTG | |
| | AAGTTGCATGCGGAGATTTTCATAATCAACCGGATTCTTATTTACATGCAC | |
| | TGCGCGAAACAGCAAAAGAAAATGACATCATCCTGACGGGCGGCTATGG | |
| | CCCTAGAGCAGAACATAATTTAGCATCACCGGATCCGGCAGTTGTTGAAA | |
| | ATGCACTTGCATTTTATAGCGAAATCTTTCGCAAAATGGATATTGCAGAC | |
| | ATACGCTCAATTGGAGGCGCACTGTATAGCTATTGGCCGATCGATTATAA | |
| | TAAAGAACTGGATAAAGATGGCGATCTTGAACGCTCAATTCAAGGAATG | |
| | CGCAAACTTGCAGATATAGCAGGACGCTATGGAATTACACTTAATATGG | |
| | AAGTTCTTAATCGCTTTGAAGGCTATCTGATCAATGATGTTCGCGAAGGA | |
| | CTGGCATATGTTAGAGCAGTTGATAAACCTAATGTTAAACTGATGCTGGA | |
| | TACGTTTCATATGAATATCGAAGAAGATTCTCTTATCGAACCGATCCTGCT | |
| | GGCAGGCAAAGATCTGGGACATGTTCATGTTGGCGAACCTAATCGCAAA | |
| | CCTCCTCGCGAAGGCCGCATGCCGTGGAAAGAAATCGGCGTTGCACTGA | |
| | GAGAAATCGGCTATGATGGAGCAGTTGTTATGGAACCGTTTGTTACGAT | |
| | GGGAGGACAAGTTGGCAAAGACATAAGAATCTGGCGCGATATTTCACAA | |
| | GGAGCAACGGAAAATGATTTAGATAGAGATGCAGCAAAAAGCGTTGCAT | |
| | TTCTGCGCTCAGCATTTGAAGAA | |
| 14 | ATGCCGTCACTTATCGGATGCCATGCATTAGTTTGGACAGGCGAATTTGA | NdeEpi1 |
| | TGCAGCAGGCATCACGAGCTCTGTTGAACGCACAAAAGCAGCAGGCTTT | |
| | GATCTGGTTGAATTTCCTCTTATGGATCCGTATACGTTTGATGCAGCAAC | |
| | GGCAAGAAAAGCACTGGGAGATGCAGATCTTACAGGAAGCGCAAGCCT | |
| | GGGACTTCCGGAACATGCAGATATTAGCTCAGAAGATCCGGCAGTTGTT | |
| | GCAGCAGGAGTTGATTTACTGAGAACGGCATTAGATAGAGTTGCAGAAA | |
| | TGGGAGGAACACGCCTGTGCGGCGTTCTGTATGGCTCAATGAGAAAACA | |
| | TATGGCACCGCTGTCAGAACGCGGACGCGCAAATAGCGTTGCAGCAATT | |
| | CGCGATCTTGCAGGCCGCGCAGCAGCAGTTGGCGTTCATCTGAGCCTGG | |
| | AAGTTGTTAATCGCTATGAAACAAATGTTTTTAATACGGGCCGCGGAGCA | |
| | TTAGCATATCTGGATGCAGTTGCAGATACGAATGTTAGCGTTCATTTAGA | |
| | TACGTATCATATGAATATCGAAGAAAGCGATTTATTTCAACCTTTTCTTGA | |
| | TACGAGCGAAAGACTTGGCTATGTTCATATTGGCGAATCACATAGAGGC | |
| | TATTTAGGAACGGGCTCAGTTGATTTTGAATCAACGTTTCGCGCACTGGC | |
| | ACGCACGGGCTATGATGGACCTATTGTTTTTGAATCGTTTTCTAGCGCAG | |
| | TTGTTAGCCCGACACTGTCTAATACACTTGGCATCTGGAGAAATCTGTGG | |
| | ACGGATTCTGAAGATCTTGCAGCACATGCAAATCGCTTTATCCGCGATCA | |
| | TATCGCAGCAGTTCGCACAATCGATTTACAT | |
| 15 | ATGCAAATCGGAGTTCATGCACTGGTTTTTACGGGAACGTTTGATAGAGA | LphEpi1 |
| | TGGCATCTCTCTGGCAGTTGAAAAAACGGCAGCAGCAGGCTTTGATCTTA | |
| | TCGAATTTCCGTTAATGGATCCGTTTGCATTTGATAGAGAACATGCAGCA | |
| | AAAGAACTTGCAGCACATGGCATCGCAGCAACGGCATCACTGGGACTGA | |
| | GCGATGCAACAGATCTGACGAGCGATGATCCGGCAGTTCGCGCAGCAG | |
| | GAGAACGCTTACTGGAAGAATGCCTGGACATAGTTCATGCATTAGGCGC | |
| | AACGAAACTGTGCGGAGTTCTGTATAGCGCAATGAAAAAATATGTTACA | |
| | CCTCCGACGGCAGAAGGCCGCGCAGCAAGCGCAGCAGCAATTGGCCGC | |
| | CTTGCAGAAAAAGCAGCAGATTTAGGAATCGATTTAAGCCTTGAAGTTGT | |
| | TAATCGCTATGAAACAAATCTTATTAATACGGGCCGCCAAGCATTAGGCT | |
| | TTCTGGATGAAATTGGACGCGGAAATGTTTTTGTTCATTTAGATACGTAT | |
| | CATATGAATATCGAAGAATCAGATCTTATTCAACCGGTTCTGGATGTTGA | |
| | TAGACGCCTGGGCTATGTTCATATCGGCGAATCACATAGAGGCTATCTGG | |
| | GCTCAGGAACAGTTGATTTTGATAGCTTTTTTCGCGGACTTGCACGCGTT | |
| | GGCTATGATGGACCGGTTGTTTTTGAATCATTTAGCTCTACGGTTGTTAG | |
| | CGCAACACTGTCTAATACACTTGGCGTTTGGAGAAATCTGTGGGATGATG | |
| | GCGATGATCTTGCAGCACATGCAAATAGATTTATTCGCGATGGACTTCGC | |
| | ACAGTTGAAACAATTGCATTACAT | |
| 16 | ATGCTTATCGGCGCACATGCATTAGTTTTTAGCGGCAGCTTTGATGCAGC | MspEpi3 |
| | AGGACTTACGTTAGCAATCGAAAAAACGAAACAAGCAGGCTTTGATCTT | |
| | ATCGAAATCCCTCTTATGGATCCGTTTCATTTTGATAGAATGCTGGCAGCA | |
| | CGCTTACTTCAAGAAAATGAACTTGCAGTTTCAGCATCACTGGGCCTGAG | |
| | CGAAGCAACAGATCTGACGTCTAATGATCCGGATGTTGTTGCAAGAGGC | |
| | GAACGCATGCTGGAAGAATGCCTGGAAATCGTTCATGCAATGGGCGGCT | |
| | CAGATCTGTGCGGAGTTATCTATTCTGCAATGAAAAAATATATGGCACCG | |
| | GCAACACCTGCAGGCCGCGCATCTAGCGCAGCAGCACTGGCACGCCTTG | |
| | CATCTCGCGCAGAAGGACTGGGTATCCGCCTGTCTCTTGAAGTTGTTAAT | |
| | CGCTATGAAACAAATTTAATGAATACGGGCCGCCAAGCACTTGCATTTCT | |
| | GGATGAAGTTGGCCATGATAATGTTAGCGTTCATTTAGATACGTATCATA | |
| | TGAATATCGAAGAAAGCGATCTGTATACACCGGTTCTTGATGTTGGCAAT | |
| | AGATTAGGATATGCACATGTTGGAGAATCACATAGAGGCTATTTAGGCT | |
| | CAGGAACGGTTGATTTTGGTTCCTTTTTTCGCGGACTGGCACGCATCGGC | |
| | TATGATGGACCGGTTGTTTTTGAATCGTTTTCTAGCGCAGTTGTTAATGAA | |
| | GAACTGTCACGCATGCTGGGAATTTGGCGCAATCTGTGGGATGATCCGG | |
| | ATGATTTAGGAGCACATGCAAATAGATTTATCAGAACACAACTGAGAGC | |
| | AGTTGAAACGATTGCATTACAT | |
| 17 | ATGAAATATGGCATCTATTATGCATATTGGGAAAAAGAATGGACGGGCG | OsiEpi1 |
| | ATTATTTTAAATATATCGATAAAGTTAAAGAACTTGGCTTTGATATTCTGG | |
| | AAATCTCTTGCGGCGCATTTAAAAAAGATTATACGACGGATGAACAACTG | |
| | CTGCGCATCGGCGAATATGCAAAAGAAAAAGGCATCATCCTTACAGCAG | |
| | GCTATGGACCTCAAAAAGATGAAAATTTAGCATCACCGGATGAAGCAGT | |
| | TCAAAAAAATGCAATCGAATTTTTTAAAGAAACACTTCGCAAACTTCATCT | |
| | TATGAATATCCATATCCTGGGAGGAGGACTGTATAGCTATTGGCCGGTTG | |
| | ATTTTAATTCTGACATAAATAAAGAACGCGATTTTGAAACGAGCGTTAAA | |
| | AATATGAGACTGATTGCAAAATATGCAGAAGAATATGATATAACATTAG | |
| | GCATGGAAGTTCTTAATCGCTTTGAAGGCTATCTGATCAATACGTGCGAT | |
| | GAATGCTTACGCTATATTGATGCAGTTGGAAATAATAGAGTTGGCGTTAT | |
| | GTTAGATACGTTTCATATGAATATCGAAGAAGATAATATCGGAGATGCAA | |
| | TTCGCAAAGCAGGAGATAAACTGGTTCATTTTCATATGGGCGAACGCAAT | |
| | CGCAAAGTTCCGGGCAAAGGCACAATGCCGTGGGCAGAAATCGGCCAA | |
| | GCACTGAGAGATATTCATTATCAACATGCAGCAGTTATGGAACCGTTTGT | |
| | TCTTCAAGGAGGCAAAGTTGGCCAAGATATTAAAATCTGGCGCAATCTGT | |
| | TACCGGATACGTCAGAAGCAAAACTGGATGCAGATGCAAAACATAGCTG | |
| | CGAATTTGTTAGACATGTTTTTGAACTT | |
| 18 | ATGCTTATTGGAGCACATGCACTGGTTTTTACGGGCTCTTTTGAACAAGC | MspEpi4 |
| | AGGACTGACGACGGCAATCGAAAAAACGAAACAAGCAGGCTTTGATTTA | |
| | ATCGAAATTCCTCTTATGGATCCGGCAAATTTTGATAGAATGCTGGCAGC | |
| | ACGCTTACTGAGAGATAATGATCTTGCAGTTAGCGCATCACTGGGCCTGT | |
| | CAGAAGCAACGGATTTAACGTCAGGCGATCCGGCAGTTGTTGCAGCAGG | |
| | AGAACGCATGCTGGAAGAATGCATCGATATTGTTCAAGCAATGGGAGGA | |
| | ACACGCCTGTGCGGAGTTATCTATAGCGCAATGAAAAAATATATGGAAC | |
| | CTGTTACGGCATCAGGACGCGCAAGCTCTGCAGCAGCATTAGGCCGCCT | |
| | TGCATCTAGAGCAGAAGATGCAGGCGTTCATCTGAGCTTAGAAGTTGTT | |
| | AATCGCTATGAAACAAATGTTATGAATACGGGCCGCCAAGCACTGGGCT | |
| | TTCTGGATGAAATTGGCCATTCTAATGTTAGCGTTCATTTAGATACGTATC | |
| | ATATGAATATCGAAGAAAGCGATCTGTATACACCGGTTCTGGATGTTGAT | |
| | ACGAGACTTGGCTATGTTCATATCGGCGAATCACATAGAGGCTATTTAGG | |
| | CTCAGGAACGGTTGATTTTGGCTCATTTTTTCGCGGACTTGCACGCGTTTC | |
| | TTATGATGGACCGGTTGTTTTTGAAAGTTTTTCTAGCGCAGTTGTTAATCA | |
| | AGATCTGTCTCGCATGCTGGGAATTTGGCGCAATCTGTGGGATGATTCAG | |
| | ATGATCTTGCAGCACATGCAAATAGATTTATTCGCGATCAACTTCGCGCA | |
| | GTTGAATCAATCTCATTACAT | |
| 19 | MKYGIYFAYWEKYWGADQVKYIKKVKELGFDILEVSGALLKNNTKERLAEIG | OspEpi5, |
| | QIAKDEGVILTCGYGPNASENLGSSDPDVVRNAISMYTDILQKMQVMDIHT | NCBI Accession |
| | FGGGIYSYWPVDYSKPIDKPGDWERSVKNVRTVGKIAEECGVDYCLEVLNRF | No. |
| | EGYLINTCEECVKFVDEVDVPSVKVMLDTFHMNIEEDDMCDAIRLAGKRLG | MBR3355450.1 |
| | HFHTGENNRKVPGRGKFPWYDLGKALREIGYDKNVVMEPFVQMGGEVGS | |
| | DIKVWRDISKGATPQQLDEDAAFSVAFLRKAFEG | |
| 20 | MKHGIYYAYWEKEWAADYLYYVEKVARLGFDLLEIGAAPLPEYSTDQIKALR | DspEpi3, |
| | DCASQNGIQLTAGYGPTYDHNMGSSDAGIRAGALEWYKRLFDVMEQLDIH | NCBI Accession |
| | LIGGALYGYWPVDFSNINKEEDWKRSVEGMHLLAPIAKEHDINLGMEVLNR | No. CDD07088.1 |
| | FESHILNTAEEGVAFVKEVGQENVKVMLDTFHMNIEEESIGDAIRTAGNLLG | |
| | HFHTGECNRMVPGKGRTPWREIGNALRDIEYDGTVVMEPFVSMGGQVGR | |
| | DIHIWRDISRGASEAELDKDAKNAVAFQKYMLDWK | |
| 21 | MKFGIYFAYWEREWNADQAKYIAKVKRLGFDGLELSCALLKNASDRELDELR | OspEpi3, |
| | RAAEAEGVTLTAGYGPAAEEDLASSDPAVTRHAVAVYTDLLKKLERLNIHTIG | NCBI Accession |
| | GGIYSYWPVDFSRPLDKAGDWARSVENVRKVGRIAGECGVNYCLECLNRFE | No. |
| | GYLINTCAECRAFVEEVDVPAVKIMLDTFHMNIEEDDMTAAIRLAGDRLGH | MBQ5973274.1 |
| | FHVGENNRRVPGKGGLPWYAVGEALRSIGYNGNVVMEPFVRPGGGVGRD | |
| | IRIWRDLSAGADEAVLDADARGSVEFLRFVFSGPTGGRDPQRCAYGSQFNRI | |
| 22 | MKYGIYFAYWEKYWDVDQVKYIKKVKDLGFDILEVSGALLKNNTRERLREIG | OspEpi10, |
| | EMAKAEGVILTCGYGPNAKEDLGSADPEVRKNAVAVYTDLLEKMRIMNIHT | NCBI Accession |
| | FGGGIYSYWPVDYSKPIDKEGDWARSVDGVRTVGKIAGECGVDYCLEVLNR | No. |
| | FEGYLINTCEECMKFVDEVGEPAVKVMLDTFHMNIEEDDMIKAIRLAGDKL | MBR2672832.1 |
| | GHFHVGENNRKLPGKGRFPWREIGRALQDIGYQKNVVMEPFVQMGGEVG | |
| | SDIKVWRDISGGATGEQLDRDAKDSVAFLRYAFEGTGGRLW | |
| 23 | MKYGIYYAYWEKEWGGDFIPYVAKVKQLGFDVLEVACGDFHNQPDSYLHA | Cb8Epi |
| | LRETAKENDIILTGGYGPRAEHNLASPDPAVVENALAFYSEIFRKMDIADIRSI | |
| | GGALYSYWPIDYNKELDKDGDLERSIQGMRKLADIAGRYGITLNMEVLNRFE | |
| | GYLINDVREGLAYVRAVDKPNVKLMLDTFHMNIEEDSLIEPILLAGKDLGHV | |
| | HVGEPNRKPPREGRMPWKEIGVALREIGYDGAVVMEPFVTMGGQVGKDI | |
| | RIWRDISQGATENDLDRDAAKSVAFLRSAFE | |
| 24 | GTGAAATATGGCATTTATTTTGCATATTGGGAAAAATATTGGGGCGCAG | OspEpi5 |
| | ATCAGGTGAAATATATTAAAAAAGTCAAAGAACTGGGCTTTGATATTCTG | |
| | GAAGTTAGCGGCGCGCTGCTGAAAAATAATACAAAAGAAAGACTGGCG | |
| | GAAATTGGCCAAATTGCAAAAGATGAAGGCGTGATTCTGACATGCGGCT | |
| | ATGGCCCGAATGCTAGCGAAAATCTTGGCAGCAGCGATCCGGATGTGGT | |
| | TAGAAATGCGATTAGCATGTATACAGATATTCTTCAAAAAATGCAAGTGA | |
| | TGGATATTCATACGTTTGGCGGCGGCATTTATAGCTATTGGCCGGTGGAT | |
| | TATAGCAAACCGATTGATAAACCGGGCGATTGGGAAAGAAGCGTTAAAA | |
| | ATGTCAGAACAGTTGGAAAAATTGCAGAAGAATGCGGCGTTGATTATTG | |
| | CCTGGAAGTGCTGAATAGATTTGAAGGCTATCTGATTAATACATGCGAA | |
| | GAATGCGTTAAATTTGTGGATGAAGTTGATGTTCCGAGCGTTAAAGTGAT | |
| | GCTTGATACATTTCATATGAATATTGAAGAAGATGATATGTGCGATGCGA | |
| | TTAGACTTGCAGGCAAAAGACTGGGCCATTTTCATACAGGAGAAAATAA | |
| | TAGAAAAGTGCCGGGCAGAGGCAAATTTCCGTGGTATGATCTTGGCAAA | |
| | GCACTTAGAGAAATTGGCTATGATAAAAATGTGGTTATGGAACCGTTTGT | |
| | GCAAATGGGCGGCGAAGTTGGATCAGATATTAAAGTTTGGAGAGATATT | |
| | AGCAAAGGAGCGACACCGCAACAACTTGATGAAGATGCAGCATTTTCAG | |
| | TTGCGTTTCTGAGAAAAGCATTTGAAGGCTAA | |
| 25 | GTGAAACATGGCATTTATTATGCGTATTGGGAAAAAGAATGGGCAGCAG | DspEpi3 |
| | ATTATCTGTATTATGTTGAAAAAGTTGCGAGACTGGGCTTTGATCTGCTG | |
| | GAAATTGGCGCAGCACCGTTACCGGAATATTCAACAGATCAAATTAAAG | |
| | CACTGAGAGATTGCGCAAGCCAAAATGGCATTCAGCTGACAGCAGGCTA | |
| | TGGCCCGACATATGATCATAATATGGGCAGCTCAGATGCAGGCATTAGA | |
| | GCAGGCGCACTTGAATGGTATAAAAGACTGTTTGATGTTATGGAACAAC | |
| | TTGATATTCATTTAATTGGCGGAGCGCTGTATGGCTATTGGCCGGTTGAT | |
| | TTTTCAAATATTAATAAAGAAGAAGATTGGAAAAGAAGCGTGGAAGGCA | |
| | TGCATCTGCTGGCGCCGATTGCAAAAGAACATGATATTAATCTTGGCATG | |
| | GAAGTTCTGAATAGATTTGAATCACATATTCTGAATACAGCAGAAGAAG | |
| | GCGTGGCATTTGTTAAAGAAGTTGGCCAAGAAAATGTTAAAGTTATGTTA | |
| | GATACATTTCATATGAATATTGAAGAAGAAAGCATTGGCGATGCGATTA | |
| | GAACAGCAGGCAATCTTCTGGGCCATTTTCATACAGGCGAATGCAATAG | |
| | AATGGTGCCGGGCAAAGGCAGAACACCGTGGAGAGAAATTGGCAATGC | |
| | ACTGAGAGATATTGAATATGATGGCACAGTCGTTATGGAACCGTTTGTTT | |
| | CAATGGGCGGCCAAGTTGGCAGAGATATTCATATTTGGAGAGATATTAG | |
| | CAGAGGCGCATCAGAAGCGGAACTGGATAAAGATGCAAAAAATGCAGT | |
| | TGCATTTCAAAAATATATGCTTGATTGGAAATAA | |
| 26 | GTGAAATTTGGCATTTATTTTGCATATTGGGAAAGAGAATGGAATGCAG | OspEpi3 |
| | ATCAAGCAAAATATATTGCAAAAGTTAAAAGACTTGGATTTGATGGCCTG | |
| | GAACTGTCATGCGCGCTGCTTAAAAATGCAAGCGATAGAGAACTGGATG | |
| | AACTTCGCAGAGCAGCGGAAGCAGAAGGCGTCACACTGACGGCAGGCT | |
| | ATGGCCCGGCGGCGGAAGAAGATCTGGCAAGCAGCGATCCGGCAGTTA | |
| | CACGCCATGCGGTGGCAGTGTATACAGATCTTCTGAAAAAACTTGAACG | |
| | CCTGAATATTCATACAATTGGAGGCGGCATTTATAGCTATTGGCCGGTGG | |
| | ATTTTAGCAGACCGCTTGATAAAGCGGGAGATTGGGCTAGAAGCGTTGA | |
| | AAATGTTAGAAAAGTTGGCAGAATTGCGGGCGAATGCGGAGTGAATTAT | |
| | TGCCTGGAATGCCTGAATCGCTTTGAAGGCTATCTGATTAATACATGCGC | |
| | AGAATGCAGAGCTTTTGTTGAAGAAGTTGATGTTCCGGCAGTGAAAATC | |
| | ATGCTGGATACATTTCATATGAATATTGAAGAAGATGATATGACGGCGG | |
| | CAATTAGACTTGCAGGCGATAGATTAGGCCATTTTCATGTGGGCGAAAAT | |
| | AATCGCAGAGTGCCGGGCAAAGGCGGCCTGCCGTGGTATGCTGTTGGC | |
| | GAAGCGCTTAGAAGCATTGGCTATAATGGCAATGTTGTTATGGAACCGTT | |
| | TGTGAGACCGGGCGGCGGCGTCGGCAGAGATATTAGAATTTGGAGAGA | |
| | TCTGAGCGCAGGCGCAGATGAAGCGGTGCTGGATGCGGATGCGAGAGG | |
| | CAGCGTGGAATTTCTGAGATTTGTTTTTAGCGGCCCGACAGGCGGCAGA | |
| | GATCCGCAGAGATGCGCGTATGGCAGCCAATTTAATAGAATTTAA | |
| 27 | GTGAAATATGGCATTTATTTCGCATACTGGGAAAAATATTGGGATGTTGA | OspEpi10 |
| | TCAAGTTAAATATATTAAGAAAGTTAAAGATCTGGGCTTCGATATTCTGG | |
| | AAGTTAGCGGCGCACTGCTGAAAAATAATACAAGAGAAAGACTGAGAG | |
| | AAATCGGCGAAATGGCAAAAGCAGAAGGCGTTATTCTGACATGCGGCTA | |
| | TGGCCCGAATGCAAAAGAGGATCTGGGCTCAGCAGATCCGGAAGTTAG | |
| | AAAAAATGCAGTTGCAGTTTATACAGATCTGCTGGAAAAAATGAGAATT | |
| | ATGAATATTCATACATTTGGCGGAGGCATTTATTCATATTGGCCGGTTGA | |
| | TTATTCAAAACCGATTGATAAAGAAGGCGATTGGGCAAGATCAGTTGAT | |
| | GGCGTTAGAACAGTTGGCAAAATTGCGGGCGAATGCGGCGTTGATTATT | |
| | GCCTGGAAGTTCTGAATAGATTTGAAGGCTATCTGATTAATACATGCGAA | |
| | GAATGCATGAAATTTGTTGATGAAGTTGGCGAACCGGCAGTTAAAGTTA | |
| | TGCTGGATACATTTCATATGAATATTGAAGAAGATGATATGATTAAAGCA | |
| | ATTAGACTGGCGGGCGATAAACTGGGCCATTTTCATGTTGGCGAAAATA | |
| | ATAGAAAACTGCCGGGCAAAGGCAGATTTCCGTGGAGAGAAATTGGAC | |
| | GCGCACTGCAAGATATTGGCTATCAAAAAAATGTTGTTATGGAACCGTTT | |
| | GTTCAAATGGGCGGCGAAGTTGGCTCAGATATTAAAGTTTGGAGAGATA | |
| | TTAGCGGCGGCGCAACGGGCGAACAACTGGATAGAGATGCAAAAGATT | |
| | CAGTTGCATTTCTGAGATATGCATTTGAAGGCACGGGCGGCAGACTGTG | |
| | GTAA | |
| 28 | ATGAAATATGGCATCTATTATGCATATTGGGAAAAAGAATGGGGCGGAG | Cb8Epi |
| | ATTTTATTCCGTATGTTGCAAAAGTTAAACAATTAGGCTTTGATGTTCTTG | |
| | AAGTTGCATGCGGAGATTTTCATAATCAACCGGATTCTTATTTACATGCAC | |
| | TGCGCGAAACAGCAAAAGAAAATGACATCATCCTGACGGGCGGCTATGG | |
| | CCCTAGAGCAGAACATAATTTAGCATCACCGGATCCGGCAGTTGTTGAAA | |
| | ATGCACTTGCATTTTATAGCGAAATCTTTCGCAAAATGGATATTGCAGAC | |
| | ATACGCTCAATTGGAGGCGCACTGTATAGCTATTGGCCGATCGATTATAA | |
| | TAAAGAACTGGATAAAGATGGCGATCTTGAACGCTCAATTCAAGGAATG | |
| | CGCAAACTTGCAGATATAGCAGGACGCTATGGAATTACACTTAATATGG | |
| | AAGTTCTTAATCGCTTTGAAGGCTATCTGATCAATGATGTTCGCGAAGGA | |
| | CTGGCATATGTTAGAGCAGTTGATAAACCTAATGTTAAACTGATGCTGGA | |
| | TACGTTTCATATGAATATCGAAGAAGATTCTCTTATCGAACCGATCCTGCT | |
| | GGCAGGCAAAGATCTGGGACATGTTCATGTTGGCGAACCTAATCGCAAA | |
| | CCTCCTCGCGAAGGCCGCATGCCGTGGAAAGAAATCGGCGTTGCACTGA | |
| | GAGAAATCGGCTATGATGGAGCAGTTGTTATGGAACCGTTTGTTACGAT | |
| | GGGAGGACAAGTTGGCAAAGACATAAGAATCTGGCGCGATATTTCACAA | |
| | GGAGCAACGGAAAATGATTTAGATAGAGATGCAGCAAAAAGCGTTGCAT | |
| | TTCTGCGCTCAGCATTTGAA | |

### Example 2: Production of D-allulose 3-epimerases in Bacillus subtilis

This example demonstrates the production of D-allulose 3-epimerase proteins in liquid fermentation of B. subtilis.

The inoculum was grown in a seed flask containing LB medium. A production medium including minerals (e.g., potassium sulfate, magnesium sulfate, ferrous sulfate, citric acid), one or more carbon sources (e.g., glucose, soy flour), and a complex nitrogen source was used to produce the exemplary epimerases. The production media was pH controlled and cells were fed according to oxygen uptake rates. The D-allulose 3-epimerase protein accumulated in the broth/cells.

Various parameters were monitored during the run and included, but were not limited to: CER (carbon dioxide evolution rate), OUR (oxygen uptake rate), pH, DO (dissolved oxygen), OD (optical density).

### Example 3: Assessment of D-allulose 3-epimerase Specific Activity and Metal Cofactor Requirements

The specific activity of the exemplary D-allulose 3-epimerases described in Example 1, produced according to the methods described in Example 2, was assayed based on the release of allulose from fructose using an HPLC method. The dependence of activity on magnesium cofactor was also assessed.

Substrate solution was prepared by mixing 5 mL of fructose (200 mM in milliq water), 0.5 mL of 1 M pH 7.5 Tris-HCl buffer, 4.5 mL of milliq water, and 20 μL of 0.5 M MgSO₄ (or 20 μL of milliq water for the group with no ion addition) in a 15-mL conical tube. Serial dilutions of epimerase samples were prepared in milliq water. Each epimerase sample (10 μL) was transferred into a new microtiter plate (Agilent 5042-1385, PP) containing 90 μL of substrate solution preincubated at 50° C. for 5 min. The incubations were done at 50° C. for 15 min with shaking (650 rpm) in an iEMS incubator (ThermoFisher). The reaction was quenched by adding 100 μL of 100 mM pH 3.5 Na-acetate buffer. The quenched reaction mixture was diluted 2-fold in milliq water and filtered for allulose analysis by HPLC using an Agilent 1200 series system with a Shodex SP0810 HPLC column. The allulose standard curve was generated and used for the calculation of allulose release from the epimerase reaction.

The specific activity was calculated using the following equation:

$$Specific Activity ( Unit / mg ) = Slope ( dose response curve ) \div 15 \times 1000$$

where 1 Unit=1 μmol allulose/min.
Table E2 shows the specific activity of the tested epimerases in the presence and absence of MgSO₄. As shown in Table E2, the control epimerase AglEpi showed significant ion dependency, while exemplary epimerase samples BsuEpi1, CbaEpi17, and CbaEpi18 showed ion independency, maintaining their activity without any ion added. Exemplary epimerases OspEpi5, OspEpi10, OspEpi3, and DspEpi3 maintained at least 60% specific activity in the absence of ion, demonstrating reduced ion dependency.

TABLE E2
D-allulose 3-epimerase specific activity on fructose
after 15-min incubation at pH 7.5, 50° C.

Specific activity (U/mg)

| Gene name | No ion | Mg2+ |
|---|---|---|
| AglEpi | 8.4 | 70.1 |
| BsuEpi1 | 24.4 | 25.0 |
| CbaEpi17 | 23.0 | 23.4 |
| CbaEpi18 | 29.6 | 30.7 |
| NdeEpi1 | 4.7 | 52.8 |
| LphEpi1 | 4.0 | 37.3 |
| MspEpi3 | 9.0 | 47.7 |
| OsiEpi1 | 19.7 | 28.5 |
| MspEpi4 | 6.1 | 59.8 |
| OspEpi5 | 34.4 | 41.5 |
| OspEpi10 | 21.7 | 28.0 |
| OspEpi3 | 27.5 | 32.5 |
| DspEpi3 | 19.8 | 31.7 |

### Example 4: pH Activity Profiles of D-allulose 3-epimerases

The pH activity profiles (from 3 to 10 pH) of D-allulose 3-epimerase samples were analyzed using fructose (100 mM) as substrate. Substrate solutions were prepared by mixing 1 mL of fructose (200 mM in milliq water), 0.04 mL of 1 M glycine/sodium acetate/HEPES (pH varying from 3 to 10), 0.96 mL of milliq water, and 4 μL of 0.5 M MgSO₄. Enzyme working solutions were prepared in water at a certain dose (showing signal within linear range as per dose response curve). All the incubations were carried out at 50° C. for 15 min with shaking (650 rpm) in an iEMS incubator (ThermoFisher). The reaction was quenched by adding 100 μL of 100 mM pH 3.5 Na-acetate buffer. The allulose released was measured by following the same procedure as described in Example 3 using the HPLC method. Enzyme activity at each pH was reported as relative activity compared to enzyme activity at optimum pH where the enzyme showed maximum activity. The pH profiles of the epimerases are shown in Table E3. OsiEpi1 retained >50% of its activity at pH 5, while the control epimerase, AglEpi, retained minimal activity under the same pH condition.

TABLE E3
pH profiles of D-allulose 3-epimerases

| | Relative activity (%) | | | | | | | |
|---|---|---|---|---|---|---|---|---|
| pH | 3 | 4 | 5 | 6 | 7 | 8 | 9 | 10 |
| AglEpi | 0 | 0 | 6 | 21 | 59 | 94 | 99 | 100 |
| BsuEpi1 | 0 | 0 | 14 | 39 | 71 | 94 | 95 | 100 |
| CbaEpi17 | 7 | 0 | 34 | 90 | 99 | 100 | 97 | 78 |
| CbaEpi18 | 0 | 0 | 6 | 59 | 83 | 85 | 80 | 100 |
| NdeEpi1 | 0 | 1 | 14 | 34 | 89 | 100 | 94 | 80 |
| LphEpi1 | 0 | 0 | 4 | 38 | 100 | 96 | 73 | 64 |
| MspEpi3 | 0 | 0 | 23 | 50 | 91 | 100 | 90 | 82 |
| OsiEpi1 | 0 | 0 | 53 | 82 | 91 | 94 | 94 | 100 |
| MspEpi4 | 0 | 8 | 16 | 57 | 100 | 100 | 81 | 79 |
| OspEpi5 | 0 | 0 | 44 | 80 | 96 | 97 | 99 | 100 |
| DspEpi3 | 0 | 0 | 11 | 42 | 79 | 97 | 98 | 100 |
| OspEpi3 | 0 | 0 | 33 | 89 | 98 | 100 | 97 | 92 |
| OspEpi10 | 0 | 1 | 26 | 64 | 100 | 100 | 96 | 99 |

### Example 5: Thermostability of D-allulose 3-epimerase

The thermostability of D-allulose 3-epimerase was evaluated by pre-incubating the enzyme working solution (500 ppm) at 70° C. for 0 (control), 5, 15, 30, 60, 120 min, respectively. The epimerase residual activity was then measured by incubating 10 μL of the above enzyme working solution with 90 μL of 100 mM of fructose at pH 7.5 and 50° C. for 15 min with shaking (650 rpm) in an iEMS incubator (ThermoFisher). The reaction was quenched by adding 100 μL of 100 mM pH 3.5 Na-acetate buffer. The allulose release was measured by following the same procedure as described in Example 3 using the HPLC method. As shown in Table E4, BsuEpi1, NdeEpi1, MspEpi3, and MspEpi4 retained >70% of their activities at 70° C. after 60 min pre-incubation. MspEpi3 retained >90% of its activity after 120-min pre-incubation. The control epimerase, AglEpi, lost its activity after 30-min pre-incubation at 70° C.

TABLE E4
D-allulose 3-epimerase thermostability at 70° C.

| Gene | Residual activity (%) after different preincubation time | | | | | |
|---|---|---|---|---|---|---|
| name | 0 min | 5 min | 15 min | 30 min | 60 min | 120 min |
| AglEpi | 100 | 22 | 6 | 2 | 0 | 0 |
| BsuEpi1 | 100 | 98 | 97 | 95 | 92 | 32 |
| CbaEpi17 | 100 | 75 | 51 | 36 | 23 | 11 |
| CbaEpi18 | 100 | 37 | 25 | 14 | 3 | 0 |
| NdeEpi1 | 100 | 87 | 85 | 81 | 74 | 42 |
| LphEpi1 | 100 | 65 | 55 | 25 | 27 | 16 |
| MspEpi3 | 100 | 107 | 92 | 90 | 90 | 91 |
| OsiEpi1 | 100 | 75 | 61 | 42 | 18 | 3 |
| MspEpi4 | 100 | 91 | 90 | 89 | 87 | 64 |
| OspEpi5 | 100 | 70 | 48 | 36 | 25 | 15 |
| DspEpi3 | 100 | 27 | 11 | 4 | 0 | 0 |
| OspEpi3 | 100 | 3 | 0 | 0 | 0 | 0 |
| OspEpi10 | 100 | 19 | 10 | 2 | 2 | 0 |

### Example 6: Temperature Profiles of D-allulose 3-epimerases

The temperature profiles of exemplary D-allulose 3-epimerase were analyzed using allulose (100 mM) as substrate. Substrate solutions were prepared by mixing 5 mL of allulose (200 mM in milliq water), 0.5 mL of 1 M pH 7.5 Tris-HCl buffer, 4.5 mL of milliq water, and 20 μL of 0.5 M MgSO₄ in a 15-mL conical tube. Enzyme working solutions were prepared in water at a dose showing signal within a linear range as per the dose response curve. All incubations were carried out at temperatures from 40° C. to 90° C., respectively, for 10 min with shaking (650 rpm) in an iEMS incubator (ThermoFisher). The reaction was quenched by adding 100 μL of 100 mM pH 3.5 Na-acetate buffer. The fructose release was measured using Megazyme's D-Fructose/D-Glucose assay kit (K-FRUGL). Enzyme activity at each temperature was reported as relative activity compared to enzyme activity at optimum temperature. The temperature profiles of the epimerases are shown in Table E5. MspEpi3 showed optimal temperature at 80° C., which was 15 degree higher than the control epimerase, AglEpi. LphEpi1, MspEpi4, NdeEpi1, and BsuEpi1 maintained >80% of their activity at 80° C. NdeEpi1 maintained 96% activity at 90° C., while AglEpi only showed 50% and 18% of its activity at 80° C. and 90° C., respectively.

TABLE E5
Temperature profiles of exemplary D-allulose 3-epimerases

| Temperature | Relative activity (%) | | | | | | | |
|---|---|---|---|---|---|---|---|---|
| (° C.) | 40 | 50 | 55 | 60 | 65 | 70 | 80 | 90 |
| MspEpi3 | 44 | 54 | 65 | 67 | 79 | 79 | 100 | 71 |
| LphEpi1 | 61 | 80 | 87 | 100 | 96 | 91 | 80 | 31 |
| OsiEpi1 | 65 | 84 | 91 | 93 | 100 | 86 | 54 | 25 |
| MspEpi4 | 81 | 93 | 99 | 100 | 100 | 89 | 85 | 67 |
| CbaEpi17 | 79 | 95 | 98 | 100 | 82 | 56 | 24 | 23 |
| NdeEpi1 | 74 | 84 | 92 | 92 | 100 | 96 | 93 | 96 |
| BsuEpil | 47 | 72 | 84 | 92 | 100 | 88 | 81 | 48 |
| CbaEpi18 | 84 | 95 | 100 | 90 | 71 | 35 | 19 | 18 |
| AglEpi | 81 | 95 | 92 | 92 | 100 | 86 | 50 | 18 |
| OspEpi5 | 53 | 69 | 81 | 90 | 100 | 77 | 27 | 14 |
| DspEpi3 | 40 | 48 | 62 | 72 | 100 | 74 | 28 | 14 |
| OspEpi3 | 70 | 93 | 100 | 77 | 33 | 19 | 10 | 7 |
| OspEpi10 | 46 | 75 | 93 | 100 | 79 | 41 | 14 | 9 |

### Example 7: Conversion of Fructose to Allulose by Immobilized D-allulose 3-epimerases

The ability of the exemplary epimerases described in Example 1 and produced according to Example 2 to convert fructose to allulose when immobilized on a matrix was assessed.

Exemplary epimerases present in lysed production broth (see, Example 2), were immobilized on granules using known crosslinking methods (U.S. Pat. No. 4,355,105). Briefly, lysed production broth was added to a slurry containing bentonite (Cholino, Patagonia, Argentina; P/N F30), Celite 505™ (Imerys), polyethyleneimine (Epomin P-1050, Nippon Shokubai) and glutaraldehyde solution 5% (Sigma Aldrich) pH adjusted to 7.3-7.6. A second addition of polyethyleneimine and glutaraldehyde 5% was then added to the slurry and pH adjusted to 8.3-8.5. Insoluble immobilized solids were filtered, extruded, and then dried using a fluid bed coater (FL-1 Fluid bed coater, Freund Vector Corporation).

To determine the activity of the immobilized epimerases, granules having immobilized epimerases (0.3 g) were pre-washed in 50 ml DI water for 30 min in a 50 ml conical tube (3-4 times). The wet granules were packed into a 1 cm×5 cm stainless steel HPLC column and column(s) were put online into an Agilent LC column compartment.

Heated 35 mM NaPO4, pH 7.5 and 5% fructose was pumped to the column at a speed of 0.2 ml/min and the column compartment was kept at 50° C. throughout the experiment. Allulose conversion efficiency was determined over the course of 14 days by HPLC, and the conversion activity at days 7 and 14 relative to day 1 (day 1=100%) is shown in Table E6 below.

As can be seen in Table E6, the conversion activity of the immobilized exemplary epimerases, CbaEpi17 and Cb8Epi, was maintained for at least 14 days. The activity of the control immobilized epimerase, however, was decreased by about 4% at day 7 compared to day 1, and exhibited a further decrease compared to day 1 at day 14.

Table E6: Percent residual activity

| Time (day) | CbaEpi17 | Cb8Epi | AglEpi (control) |
|---|---|---|---|
| 7 days | 101% | 101% | 96% |
| 14 days | 101% | 102% | 93% |

These data suggest that the immobilized exemplary epimerases are able to maintain enzymatic activity over time.

### Example 8: Conversion of Fructose to Allulose by Immobilized D-allulose 3-epimerases Under Stress Conditions

The ability of the exemplary epimerases described in Example 1 and produced according to Example 2 to convert fructose to allulose when immobilized on a matrix was assessed under various temperature and pH stress conditions and compared against the soluble enzyme.

Matrix immobilized enzyme was prepared from lysed production broth according to Example 7. Soluble enzyme was isolated from lysed production broth (see, Example 2) using known purification methods. Briefly, the soluble components of lysed production broth were loaded onto a Phenyl Sepharose FF column equilibrated with 20 mM Tris pH 7.0 and 1M ammonium sulfate. The column was washed with equilibration buffer and eluted with 20 mM Tris pH 7.0. The epimerase containing fractions where then loaded onto a Q-Sepharose column equilibrated with 20 mM Tris pH 7.0 and 150 mM NaCl. The column was washed with equilibration buffer and eluted with 20 mM Tris pH 7.0 and 1M NaCl. The epimerase containing fractions where then concentrated with a Sartorius Vivaflow 200 Crossflow Cassette equipped with a 10 kDa MWCO membrane. The purified epimerase was formulated with 12 mM Tris pH 7.0, 90 mM NaCl, and 40% Glycerol.

To assess activity under various conditions, 8 μg of soluble enzyme or 5 mg immobilized enzyme granules were loaded into the wells of a microtiter plate and combined with 200 μL of a 50% fructose syrup containing 1 mM MgSO₄, 100 mM NaCl, 1 mM Sodium Metabisulfite, and 20 mM of either sodium phosphate buffer (pH 7.2) or sodium acetate buffer (pH 5.0). Prior to adding the syrup, enzymes were subjected to a stress condition of either 20° C. or 60° C. in either sodium phosphate buffer (pH 7.2) or sodium acetate buffer (pH 5.0) for 1 hour. Epimerase was then incubated at 50° C. for 1 hour with the fructose syrup to measure epimerase activity. Activity was determined by fructose conversion as measured by HPLC. Conversion relative to the control condition (20° C., pH 7.2) is shown for both soluble and immobilized exemplary enzyme Cb8Epi in Table E7.

TABLE E7
Residual epimerase activity of soluble and
immobilized enzyme under various conditions

| | | Stress Temp | | Activity Relative to |
|---|---|---|---|---|
| Enzyme Format | pH | (° C.) | % Allulose | Control Condition |
| Immobilized | 7.2 | 20 | 4.5% | 100% |
| Immobilized | 7.2 | 60 | 4.3% | 98% |
| Immobilized | 5 | 60 | 4.1% | 97% |
| Immobilized | 5 | 20 | 4.0% | 96% |
| Soluble | 7.2 | 20 | 9.4% | 100% |
| Soluble | 7.2 | 60 | 0.4% | 4% |
| Soluble | 5 | 60 | 0.0% | 0% |
| Soluble | 5 | 20 | 0.0% | 0% |

These data show the soluble enzyme maintains activity at pH 7.2 and 20° C. while the immobilized enzyme maintains activity under both thermal and low pH stress conditions.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. Although the invention may be described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A protein comprising an amino acid sequence having at least 70%, 80%, 90%, 95%, 99%, or 100% sequence identity to the sequence set forth by SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:19, SEQ ID NO:20, SEQ ID NO:21, SEQ ID NO:22, or SEQ ID NO: 23, wherein the protein has epimerase activity.

2. (canceled)

3. The protein of claim 1, wherein the protein is immobilized on a matrix.

4. The protein of claim 3, wherein the matrix is a granule or an ion exchange resin.

5. A nucleic acid molecule comprising a nucleic acid sequence encoding a protein according to claim 1.

6. The nucleic acid molecule of claim 5, comprising a nucleic acid sequence that:
i) encodes an amino acid sequence having at least 90%, 95%, 99%, or 100% sequence identity to the sequence set forth by SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 19, SEQ ID NO:20, SEQ ID NO:21, SEQ ID NO:22, or SEQ ID NO:23;
ii) has at least 70%, 80%, 90%, 95%, 99%, or 100% sequence identity to the sequence set forth by SEQ ID NO:11, SEQ ID NO:12, SEQ ID NO:13, SEQ ID NO:14, SEQ ID NO: 15, SEQ ID NO:16, SEQ ID NO:17, SEQ ID NO:18, SEQ ID NO:24, SEQ ID NO:25, SEQ ID NO:26, SEQ ID NO:27, or SEQ ID NO:28; or
iii) hybridizes under stringent conditions to a nucleic acid sequence having a sequence complementary to the sequence set forth by SEQ ID NO:11, SEQ ID NO:12, SEQ ID NO: 13, SEQ ID NO:14, SEQ ID NO:15, SEQ ID NO:16, SEQ ID NO:17, or SEQ ID NO:18, SEQ ID NO:24, SEQ ID NO:25, SEQ ID NO:26, SEQ ID NO:27, or SEQ ID NO:28.

7. The nucleic acid molecule of claim 5, comprising a heterologous regulatory sequence.

8. A vector comprising a nucleic acid molecule according to claim 5.

9. A host cell comprising a nucleic acid molecule according to claim 5.

10. The host cell of claim 9, wherein the host cell is a yeast, a bacterium, a mammalian cell, or a plant cell.

11. The host cell of claim 9, wherein the host cell is a Bacillus spp.

12. A cultured cell material comprising a protein of claim 1.

13. (canceled)

14. A composition for producing allulose comprising:
i) a protein according to claim 1; and
ii) a substrate comprising fructose.

15.-20. (canceled)

21. A method of producing allulose, comprising contacting a protein according to claim 1 with a substrate comprising fructose.

22. The method of claim 21, wherein the contacting occurs under conditions comprising a temperature in a range of about 50° C. to about 90° C.

23. The method of claim 21, wherein the contacting occurs under conditions comprising a pH in a range of about 4.5 to about 8.

24. The method of claim 21, wherein the contacting occurs under conditions where no metal cofactor is added or an amount of metal cofactor that is less than a metal cofactor concentration needed for epimerase activity is added.

25. (canceled)

26. The method of claim 21, wherein the substrate comprising fructose is produced by:
(i) contacting a substrate comprising glucose with a glucose isomerase prior to contacting the protein; or
(ii) contacting a substrate comprising glucose with a glucose isomerase at the same time as contacting the protein.

27. The method of claim 21, comprising purifying the produced allulose.

28. (canceled)

29. The method of claim 21, wherein the protein is comprised in a cultured cell material.
